(19) United States
(12) Patent Application Publication
(10) Pub. No.: US 2011/0150796 A1
(43) Pub. Date: Jun. 23, 2011

(54) PRECIPITATION POLYMERIZATION IN THE PRESENCE OF GLYCERIN MONOSTEARATE

(75) Inventors: Son Nguyen KIM, Hemsbach (DE); Wolfgang Jahnel, Bellheim (DE)

(73) Assignee: BASF SE, Ludwigshafen (DE)

(21) Appl. No.: 13/062,197

(22) PCT Filed: Sep. 3, 2009
(86) PCT No.: PCT/EP2009/061377

§ 371 Date: Mar. 3, 2011

(30) Foreign Application Priority Data
Sep. 4, 2008 (EP) 08163686.2

(51) Int. Cl. A61K 8/81; C08F 2/06; C08L 33/02; A61Q 19/00; A61Q 3/00; A61Q 1/04; A61Q 1/10; A61Q 9/00; A61Q 5/08; A61K 47/32; A61P 17/10; A61P 31/00; A61Q 5/02; A61K 8/96; A61Q 19/10; A21D 2/16

(52) U.S. Cl. 424/59; 526/216; 526/209; 524/560; 514/772.6; 424/61; 424/64; 424/70.7; 424/73; 424/62; 510/119; 510/130; 510/158; 426/654

(57) ABSTRACT

A process for preparing a copolymer composition A) by free-radical copolymerization of a monomer composition comprising: a) acrylic acid, b) optionally at least one compound, different from a), having a free-radically polymerizable, α,β-ethylenically unsaturated double bond and at least one anionogenic and/or anionic group per molecule, c) at least one free-radically polymerizable crosslinking compound which comprises at least two α,β-ethylenically unsaturated double bonds per molecule. The process is performed by precipitation polymerization in the presence of an auxiliary composition H) comprising H1) glycerol monostearate, and H2) at least one compound with an HLB value in the range from 4 to 10, selected from water-insoluble natural waxes, nonionic emulsifiers and mixtures thereof.

The present invention relates to a process for the preparation of a crosslinked copolymer having anionogenic/anionic groups by free-radical copolymerization according to the method of precipitation polymerization, to the copolymers obtained by this process and to their use.

Rheology modifiers, which are often processed in solid, pulverulent form, are used in many technical fields, e.g. coatings, paper production, textile industry, hygiene products, cosmetic and pharmaceutical compositions. The rheology modifiers currently used most often include crosslinked polyacrylic acids.

U.S. Pat. No. 3,915,921 describes copolymers which comprise, in copolymerized form, an olefinically unsaturated carboxylic acid, a C₁₀-C₃₀-alkyl (meth)acrylate and optionally a crosslinking monomer having at least 2 ethylenically unsaturated double bonds. In neutralized form, they serve as thickeners for various applications.

U.S. Pat. No. 2,798,053 describes copolymers of acrylic acid and polyethers having at least two allyl groups per molecule.

WO 2007/010034 describes an ampholytic copolymer A) obtainable by free-radical copolymerization of
a) at least one compound having a free-radically polymerizable, α,β-ethylenically unsaturated double bond and at least one anionogenic and/or anionic group per molecule,
b) at least one compound which is selected from N-vinylimidazole compounds, N-[3-(dimethylamino)propyl]acrylamide, N-[3-(dimethylamino)propyl]methacrylamide and mixtures thereof, and
c) at least one free-radically polymerizable crosslinking compound which comprises at least two α,β-ethylenically unsaturated double bonds per molecule.

WO 2007/012610 describes a silicone-group-containing copolymer A) obtainable by free-radical copolymerization of
a) at least one compound having a free-radically polymerizable, α,β-ethylenically unsaturated double bond and at least one ionogenic and/or ionic group per molecule,
b) at least one free-radically polymerizable crosslinking compound which comprises at least two α,β-ethylenically unsaturated double bonds per molecule,
   in the presence of at least one silicone compound c) comprising a polyether group and/or a free-radically polymerizable olefinically unsaturated double bond, where the copolymerization can take place in accordance with the method of precipitation polymerization.

WO 2007/010035 describes the use of an ampholytic copolymer which has a molar excess of anionogenic/anionic groups compared with cationogenic/cationic groups or which has a molar excess of cationogenic/cationic groups compared with anionogenic/anionic groups and which is obtainable by free-radical copolymerization of
a1) at least one compound having a free-radically polymerizable, α,β-ethylenically unsaturated double bond and at least one anionogenic and/or anionic group per molecule,
a2) at least one compound having a free-radically polymerizable, α,β-ethylenically unsaturated double bond and at least one cationogenic and/or cationic group per molecule,
b) at least one free-radically polymerizable crosslinking compound which comprises at least two α,β-ethylenically unsaturated double bonds per molecule,
c) optionally in the presence of at least one silicone compound comprising a polyether group and/or a free-radically polymerizable olefinically unsaturated double bond,
   as rheology modifier for hair cosmetic compositions, where the ampholytic copolymer can be prepared by free-radical copolymerization according to the method of precipitation polymerization.

U.S. Pat. No. 4,758,641 describes a process for the preparation of polymers of olefinically unsaturated C₃-C₅-carboxylic acids in a solvent which is selected from acetone and lower alkyl acetates, and in the presence of a crosslinker.

U.S. Pat. No. 4,692,502 describes a process for the polymerization of olefinically unsaturated carboxylic acids in an organic solvent and in the presence of ionic surfactants.

U.S. Pat. No. 4,526,937 describes a process for the precipitation polymerization of olefinically unsaturated carboxylic acids in the presence of polyoxyethylene-polyoxypropylene block copolymers with terminal OH groups and an HLB value of greater than 10.

U.S. Pat. No. 4,419,502 describes a process for the precipitation polymerization of olefinically unsaturated carboxylic acids in methylene chloride and in the presence of a polyoxyethylene alkyl ether and/or polyoxyethylene sorbitol ester having an HLB value of greater than 12.

EP 0 584 771 A1 describes a polymer of an olefinically unsaturated carboxylic acid and a steric stabilizer. Suitable steric stabilizers are linear block copolymers and random comb polymers with hydrophilic and hydrophobic units.

U.S. Pat. No. 4,375,533 describes a process for the polymerization of olefinically unsaturated carboxylic acids in a polymerization medium in which the carboxylic acid polymer is insoluble, in the presence of a surfactant having an HLB value of less than 10. Besides a large multitude of further surfactants, glycerol monostearate is mentioned. However, it is neither described to use this in the form of mixtures, nor is it used in the working examples.

U.S. Pat. No. 4,420,596 describes a process for the polymerization of olefinically unsaturated carboxylic acids in a polymerization medium which comprises petroleum spirit in the presence of 1) a sorbitan ester, 2) a nonionic surfactant having an HLB value of less than 10, which is an ester of glycerol or an alkylene glycol and 3) a long-chain alcohol. Besides a large multitude of further surfactants, glycerol monostearate is mentioned. However, it is neither described to use this in the form of mixtures, nor is it used in the working examples.

EP 1 209 198 A1 describes a polymer composition which comprises A) a crosslinked carboxyl-group-containing polymer and B) at least one compound which is selected from esters of polyhydric alcohols with fatty acids and the alkylene oxide adducts thereof. Among a large multitude of various compounds B), glycerol monostearate is also mentioned. However, preference is given to using decaglyceryl decaoleate, decaglyceryl pentaoleate, decaglyceryl diisostearate, decaglyceryl oleate, hexaglyceryl oleate, tetraglyceryl stearate, diglyceryl oleate and glyceryl trioleate (paragraph [0023]). The polymer composition serves as thickener for various aqueous solutions.

There is still a need for polymers which are highly suitable for adjusting the rheological properties of various products so that they can be formulated, for example, in the form of gels. The gels obtained here should be characterized by at least one of the following properties: very good clarity, very good structure, good dissolution properties. In this connection, the polymerization should be able to be carried out as far as possible without undesired complications, such as an excessive increase in the viscosity or undesired deposit formation in the polymerization reactor. The polymers obtained should be characterized, for example, by high uniformity.

Surprisingly, it has now been found that this object is achieved by a polymerization process where the preparation of a crosslinked copolymer having anionogenic and/or anionic groups takes place by free-radical copolymerization according to the method of precipitation polymerization in the presence of an auxiliary composition which comprises glycerol monostearate and at least one compound different therefrom having an HLB value in the range from 4 to 10.

The invention therefore firstly provides a process for the preparation of a copolymer composition A) by free-radical copolymerization of a monomer composition comprising
a) acrylic acid,
b) optionally at least one compound, different from a), having a free-radically polymerizable, α,β-ethylenically unsaturated double bond and at least one anionogenic and/or anionic group per molecule,
c) at least one free-radically polymerizable crosslinking compound which comprises at least two α,β-ethylenically unsaturated double bonds per molecule,
   by the method of precipitation polymerization in the presence of an auxiliary composition H) comprising
H1) glycerol monostearate, and
H2) at least one compound, different from H1), with an HLB value in the range from 4 to 10, selected from water-insoluble natural waxes, nonionic emulsifiers and mixtures thereof.

In a specific embodiment, for the preparation of the copolymer composition A), additionally at least one further auxiliary H3) is used which is selected from emulsifiers with an HLB value in the range greater than 10.

The invention further provides a copolymer composition A) which is obtainable by this process.

The use of the auxiliary system according to the invention for the preparation of A) by precipitation polymerization brings with it at least one of the following advantages:
the reaction mixture has a lower viscosity, meaning that the heat of reaction can be better dissipated;
in the case of the reaction and/or the formulations, higher solids contents are possible;
deposit formation in the polymerization reactor can generally be successfully avoided;
the lower viscosity and/or the high solids contents render the process more economical;
the gels obtained are characterized by at least one of the following properties: very good clarity, very good structure, good dissolution properties.

Within the context of the present invention, the HLB value (hydrophilic lipophilic balance) is used, inter alia, to characterize the properties of the auxiliaries H) used. A definition of the HLB value can be found in W. C. Griffin, J. Soc. Cosmetic Chem., volume 5,249 (1954). For such nonionic emulsifiers and also for ionic emulsifiers for which the HLB value cannot be calculated, an experimental determination by the emulsion comparison method is often successful. The HLB value of the unknown emulsifier is calculated according to the formula:

$$H L B = \frac{R - ( H \times S )}{N}$$

R=the required HLB value of the oil, which must be known,
H=the HLB value of the known emulsifier,
S=weight percent of the known emulsifier, expressed as a decimal (e.g. 45%=0.45),
N=weight percent of the emulsifier whose HLB is to be ascertained, expressed as a decimal.

HLB values and methods for their determination are described in standard works, e.g. K. H. Schrader, Grundlagen and Rezepturen der Kosmetika [Fundamentals and formulations of cosmetics], Hüthig Verlag, 2nd edition, 1989.

The preparation of the copolymer compositions A) according to the invention takes place by precipitation polymerization. In the precipitation polymerization, the monomers used are soluble in the reaction medium (monomer, solvent) but the corresponding polymer is not. The polymer which forms becomes insoluble under the selected polymerization conditions and precipitates out of the reaction mixture. The process according to the invention itself is characterized by advantageous properties and, moreover, also leads to copolymer compositions with particularly advantageous properties. Thus, an undesirably large increase in the viscosity of the reaction medium does not result during the polymerization. Deposit formation can usually be successfully avoided. The precipitation polymers present in the polymer compositions according to the invention are characterized by their capability as rheology modifiers (specifically as thickeners). They are suitable for the formulation of gels with improved clarity and/or improved structural properties and/or improved dissolution properties compared with gels based on conventional polymer compositions.

Within the context of the present invention, the expression alkyl comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 1-methylbutyl, 2-methylbutyl, 3-methylbutyl, 1,2-dimethylpropyl, 1,1-dimethylpropyl, 2,2-dimethylpropyl, 1-ethylpropyl, n-hexyl, 1-methylpentyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, etc.

Suitable relatively long-chain C₈-C₃₀-alkyl groups and C₈-C₃₀-alkenyl groups are straight-chain and branched alkyl and alkenyl groups. These are preferably predominantly linear alkyl radicals, as also occur in natural or synthetic fatty acids and fatty alcohols and also oxo alcohols, or are predominantly linear alkenyl radicals as also occur in natural or synthetic fatty acids and fatty alcohols and also oxo alcohols, which may be mono-, di- or polyunsaturated. Suitable relatively long-chain C₈-C₃₀-alkyl groups are, for example, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, arachinyl, behenyl, lignocerinyl, melissinyl, etc. Suitable relatively long-chain C₈-C₃₀-alkenyl groups comprise, for example, n-octenyl, n-nonenyl, n-decenyl, n-undecenyl, n-dodecenyl, n-tridecenyl, n-tetradecenyl, n-pentadecenyl, n-hexadecenyl, n-heptadecenyl, n-octadecenyl, n-nonadecenyl, n-eicosenyl, n-docosenyl, n-tetracosenyl, hexacosenyl, triacontenyl, etc.

Cycloalkyl is preferably C₅-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

In the text below, compounds which can be derived from acrylic acid and methacrylic acid are sometimes referred to for short by adding the syllable “(meth)” to the compound derived from acrylic acid.

The copolymer compositions A) according to the invention can advantageously be formulated as gels under normal conditions (20° C.). “Gel-like consistency” indicates formulations which have a higher viscosity than a liquid and which are self-supporting, i.e. which retain a shape imparted to them without shape-stabilizing covering. In contrast to solid formulations, gel-like formulations, however, can be readily deformed under the application of shear forces. The viscosity of the gel-like compositions is preferably in a range from greater than 600 to about 60 000 mPas, particularly preferably from 6000 to 30 000 mPas. The gels are preferably hair gels.

Within the context of the present invention, water-soluble monomers and polymers are to be understood as meaning monomers and polymers which dissolve in water to at least 1 g/l at 20° C. Water-dispersible monomers and polymers are to be understood as meaning monomers and polymers which disintegrate into dispersible particles under application of shear forces, for example by stirring. Hydrophilic monomers are preferably water-soluble or at least water-dispersible. The copolymers present in the copolymer compositions A) according to the invention are generally water-soluble.

### Monomer a)

To prepare the copolymer compositions A) according to the invention, acrylic acid is used as component a). Component a) is preferably used in an amount of at least 60% by weight, based on the total weight of the compounds used for the polymerization. Component a) is particularly preferably used in an amount of from 60 to 99.9% by weight, based on the total weight of the compounds used for the polymerization (i.e. monomers a) and, if present, b) to g) add up to 100% by weight).

In a first preferred embodiment, the monomer composition used for the preparation of the copolymer composition A) by free-radical copolymerization consists only of components a) and c). Component a) is then preferably used in an amount of from 95 to 99.99% by weight, particularly preferably from 98 to 99.9% by weight, based on the total weight of the compounds a) and c) used for the polymerization. The process according to the invention then serves specifically for the preparation of crosslinked polyacrylic acid.

In a second preferred embodiment, the component a) is used in an amount of from 25 to 99.7% by weight, preferably 30 to 99.5% by weight, based on the total weight of the compounds used for the polymerization (i.e. components a) to g)). In this embodiment, the monomer composition used for the preparation of the copolymer composition A) also comprises at least one further monomer besides the components a) and c).

### Monomer b)

In the process according to the invention for the preparation of the copolymer composition A), a compound different from acrylic acid having a free-radically polymerizable, α,β-ethylenically unsaturated double bond and at least one anionogenic and/or anionic group per molecule can optionally be used as component b). The component b) is preferably used in an amount of from 0 to 40% by weight, particularly preferably from 0 to 25% by weight, based on the total weight of the compounds used for the polymerization. If present, the component b) is preferably used in an amount of from 0.1 to 40% by weight, particularly preferably from 0.5 to 25% by weight, based on the total weight of the compounds used for the polymerization.

The compounds b) are preferably selected from monoethylenically unsaturated carboxylic acids, sulfonic acids, phosphonic acids and mixtures thereof.

The monomers b) include monoethylenically unsaturated mono- and dicarboxylic acids having 3 to 25, preferably 3 to 6, carbon atoms, which can also be used in the form of their salts or anhydrides. Examples thereof are methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a) further include the half-esters of monoethylenically unsaturated dicarboxylic acids having 4 to 10, preferably 4 to 6, carbon atoms, e.g. of maleic acid, such as monomethyl maleate. The monomers a) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers a) also include the salts of the aforementioned acids, in particular the sodium, potassium and ammonium salts and also the salts with amines. The monomers b) can be used as they are or as mixtures with one another. The stated weight fractions all refer to the acid form.

The component b) is preferably selected from methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

Methacrylic acid is particularly preferably used as component b).

### Crosslinker c)

To prepare the copolymer compositions A), at least one crosslinker, i.e. a compound with two or more than two ethylenically unsaturated, nonconjugated double bonds, is used according to the invention.

Preferably, crosslinkers are used in an amount of from 0.01 to 5% by weight, particularly preferably 0.1 to 4% by weight, based on the total weight of the compounds used for the polymerization.

Suitable crosslinkers c) are, for example, acrylic esters, methacrylic esters, allyl ethers or vinyl ethers of at least dihydric alcohols. The OH groups of the parent alcohols here may be completely or partially etherified or esterified; however, the crosslinkers comprise at least two ethylenically unsaturated groups.

Examples of the parent alcohols are dihydric alcohols such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiapentane-1,5-diol, and also polyethylene glycols, polypropylene glycols and polytetrahydrofurans with molecular weights of in each case 200 to 10 000. Apart from the homopolymers of ethylene oxide and propylene oxide, it is also possible to use block copolymers of ethylene oxide or propylene oxide or copolymers which comprise ethylene oxide and propylene oxide groups in incorporated form. Examples of parent alcohols having more than two OH groups are trimethylolpropane, glycerol, pentaerythritol, 1,2,5-pentanetriol, 1,2,6-hexanetriol, cyanuric acid, sorbitans, sugars such as sucrose, glucose, mannose. The polyhydric alcohols can of course also be used following reaction with ethylene oxide or propylene oxide as the corresponding ethoxylates or propoxylates. The polyhydric alcohols can also firstly be converted to the corresponding glycidyl ethers by reaction with epichlorohydrin. Preference is given to ethylene glycol di(meth)acrylate and polyethylene glycol di(meth)acrylates.

Further suitable crosslinkers c) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellal, crotyl alcohol or cis-9-octadecen-1-ol. However, it is also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers c) are esters, different from (meth)acrylates, of unsaturated carboxylic acids with the polyhydric alcohols described above, for example oleic acid, crotonic acid, cinnamic acid or 10-undecenoic acid.

Suitable as crosslinker c) are, moreover, straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable as crosslinker c) are the acrylamides, methacrylamides and N-allylamines of at least difunctional amines. Such amines are, for example, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid or at least dibasic carboxylic acids, as have been described above.

Also suitable as crosslinker c) are triallylamine and triallylmonoalkylammonium salts, e.g. triallylmethylammonium chloride or methylsulfate.

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers c) are divinyldioxane, tetraallylsilane or tetravinylsilane.

Mixtures of the aforementioned compounds c) can of course also be used.

Very particularly preferred crosslinkers c) are ethylene glycol di(meth)acrylate, polyethylene glycol di(meth)acrylates, pentaerythritol triallyl ether, methylenebisacrylamide, N,N′-divinylethyleneurea, triallylamine and triallylmonoalkylammonium salts.

### Basic Monomer d)

The monomer composition used for the preparation of the copolymer composition A) can additionally comprise at least one compound d) with a free-radically polymerizable, α,β-ethylenically unsaturated double bond and at least one cationogenic and/or cationic group per molecule.

The component d) is preferably used in an amount of from 0.1 to 50% by weight, particularly preferably 0.2 to 25% by weight, based on the total weight of the compounds used for the polymerization.

Preferably, the copolymers present in the copolymer composition A) have an excess of anionogenic and/or anionic groups. Consequently, if monomers d) are used, then it is preferably in amounts such that the copolymer in A) has a molar excess of anionogenic/anionic groups compared with cationogenic/cationic groups of at least 5:1, preferably at least 10:1.

The cationogenic and/or cationic groups of component d) are preferably nitrogen-containing groups, such as primary, secondary and tertiary amino groups and also quaternary ammonium groups. The nitrogen-containing groups are preferably tertiary amino groups or quaternary ammonium groups. Charged cationic groups can be produced from the amine nitrogens either by protonation or by quaternization with acids or alkylating agents. These include, for example, carboxylic acids, such as lactic acid, or mineral acids, such as phosphoric acid, sulfuric acid and hydrochloric acid, or as alkylating agents C₁-C₄-alkyl halides or sulfates, such as ethyl chloride, ethyl bromide, methyl chloride, methyl bromide, dimethyl sulfate and diethyl sulfate. A protonation or quaternization can generally take place either before or after the polymerization.

The component d) is preferably selected from esters of α,β-ethylenically unsaturated mono- and dicarboxylic acids with amino alcohols, which may be mono- or dialkylated on the amine nitrogen, amides of α,β-ethylenically unsaturated mono- and dicarboxylic acids with diamines which have at least one primary or secondary amino group, N,N-diallylamine, N,N-diallyl-N-alkylamines and derivatives thereof, vinyl- and allyl-substituted nitrogen heterocycles, vinyl- and allyl-substituted heteroaromatic compounds, and mixtures thereof.

Preferred compounds d) are the esters of α,β-ethylenically unsaturated mono- and dicarboxylic acids with amino alcohols. Preferred amino alcohols are C₂-C₁₂-amino alcohols which are C₁-C₈-mono- or -dialkylated on the amine nitrogen. Of suitability as acid component of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof as acid component.

Preferred monomers d) are N-methylaminoethyl (meth)acrylate, N-ethylaminoethyl (meth)acrylate, N-(n-propyl)aminoethyl (meth)acrylate, N-(tert-butyl)aminoethyl (meth)acrylate, N,N-dimethylaminomethyl (meth)acrylate, N,N-dimethylaminoethyl (meth)acrylate, N,N-diethylaminomethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate.

Particular preference is given to N,N-dimethylaminoethyl acrylate, N,N-dimethylaminoethyl methacrylate and mixtures thereof. Preferred monomers d) are in particular also the quaternization products of the aforementioned compounds.

In one very specific embodiment, the component d) consists only of N,N-dimethylaminoethyl (meth)acrylate or N,N-dimethylaminoethyl (meth)acrylate quaternized with methyl chloride or dimethyl sulfate.

Preferred monomers d) are also N,N-diallylamines and N,N-diallyl-N-alkylamines and their acid addition salts and quaternization products. Alkyl here is preferably C₁-C₂₄-alkyl. Preference is given to N,N-diallyl-N-methylamine and N,N-diallyl-N,N-dimethylammonium compounds, such as, for example, the chlorides and bromides. Particular preference is given to N,N-diallyl-N-methylammonium chloride (DADMAC).

Suitable monomers d) are also the amides of the aforementioned α,β-ethylenically unsaturated mono- or dicarboxylic acids with diamines which have at least one primary or secondary amino group. Preference is given to diamines which have one tertiary and one primary or secondary amino group.

Preferred monomers d) are, for example, N-[tert-butylaminoethyl](meth)acrylamide, N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide.

In one suitable embodiment, the component d) comprises at least one N-vinylimidazole compound as vinyl-substituted heteroaromatic compound. In one specific embodiment, component d) is selected from N-vinylimidazole compounds and mixtures which comprise at least one N-vinylimidazole compound.

Suitable N-vinylimidazole compounds are compounds of the formula

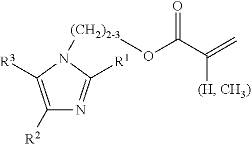

in which R¹ to R³, independently of one another, are hydrogen, C₁-C₄-alkyl or phenyl. Preferably, R¹ to R³ are hydrogen.

Also suitable are N-vinylimidazole compounds of the general formula (I)

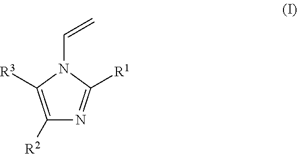

in which R¹ to R³, independently of one another, are hydrogen, C₁-C₄-alkyl or phenyl.

Examples of compounds of the general formula (I) are given in table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

As monomer d), preference is given to 1-vinylimidazole (N-vinylimidazole) and mixtures which comprise N-vinylimidazole.

Suitable monomers d) are also the compounds obtainable by protonation or quaternization of the aforementioned N-vinylimidazole compounds. Examples of such charged monomers d) are quaternized vinylimidazoles, in particular 3-methyl-1-vinylimidazolium chloride, methosulfate and ethosulfate. Suitable acids and alkylating agents are those listed previously.

Suitable monomers d) are also vinyl- and allyl-substituted nitrogen heterocycles different from vinylimidazoles, such as 2- and 4-vinylpyridine, 2- and 4-allylpyridine, and the salts thereof.

### Monomer e)

In a further embodiment, the monomer composition used for the preparation of the copolymer composition can additionally comprise, in copolymerized form, at least one further amide-group-containing monomer e) of the general formula (II)

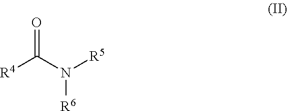

where
one of the radicals R⁴ to R⁶ is a group of the formula CH₂═CR⁷— where R⁷═H or C₁-C₄-alkyl and the other radicals R⁴ to R⁶, independently of one another, are H, alkyl, cycloalkyl, heterocycloalkyl, aryl or hetaryl,
where R⁴ and R⁵, together with the amide group to which they are bonded, may also be a lactam having 5 to 8 ring atoms,
where R⁵ and R⁶, together with the nitrogen atom to which they are bonded, may also be a five- to seven-membered heterocycle,
with the proviso that the sum of the carbon atoms of the radicals R⁴, R⁵ and R⁶ is at most 8.

Preferably, in addition to the carbonyl carbon atom of the amide group, the compounds of component e) additionally have at most 7 further carbon atoms.

The compounds of component e) are preferably selected from primary amides of α,β-ethylenically unsaturated monocarboxylic acids, N-vinylamides of saturated monocarboxylic acids, N-vinyllactams, N-alkyl- and N,N-dialkylamides of α,β-ethylenically unsaturated monocarboxylic acids and mixtures thereof.

Preferred monomers e) are N-vinyllactams and derivatives thereof which can have, for example, one or more C₁-C₆-alkyl substituents, such as methyl, ethyl, n-propyl, isopropyl, n-butyl, sec-butyl, tert-butyl, etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc.

Particular preference is given to using N-vinylpyrrolidone and/or N-vinylcaprolactam.

Suitable monomers e) are also acrylamide and methacrylamide.

Suitable N-alkyl- and N,N-dialkylamides of α,β-ethylenically unsaturated monocarboxylic acids which, in addition to the carbonyl carbon atom of the amide group, have at most 7 further carbon atoms are, for example, N-methyl(meth)acrylamide, N-ethyl(meth)acrylamide, N-propyl(meth)acrylamide, N-(n-butyl)(meth)acrylamide, N-tert-butyl(meth)acrylamide, n-pentyl(meth)acrylamide, n-hexyl(meth)acrylamide, n-heptyl(meth)acrylamide, N,N-dimethyl(meth)acrylamide, N,N-diethyl(meth)acrylamide, piperidinyl(meth)acrylamide, morpholinyl(meth)acrylamide and mixtures thereof.

Open-chain N-vinylamide compounds suitable as monomers e) are, for example, N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide, N-vinylbutyramide and mixtures thereof. Preference is given to using N-vinylformamide.

The component e) is preferably used in an amount of from 0.1 to 50% by weight, particularly preferably 1 to 40% by weight, based on the total weight of the monomers used for the polymerization.

### Monomer f)

In a further embodiment, the monomer composition used for the preparation of the copolymer composition can additionally comprise, in copolymerized form, at least one further monomer f) which has a group of the formulae (IIIa) or (IIIb)

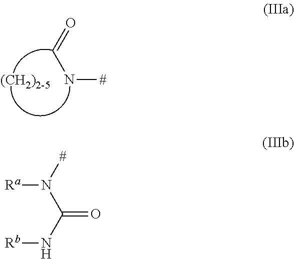

in which
# is the bonding site to a group with a free-radically polymerizable, α,β-ethylenically unsaturated double bond, where in the compounds (IIIa) # is not the bonding site to a group of the formula CH₂═CR⁷— where R⁷═H or C₁-C₄-alkyl (=monomers e),
R^{a} is H or C₁-C₄-alkyl,
R^{b} is H or C₁-C₄-alkyl, or
R^{a} and R^{b} together are (CH₂)₁₋₄.

Preferably, the monomer f) is selected from monomers having a group of the formulae (IIIa.1) or (IIIb.1)

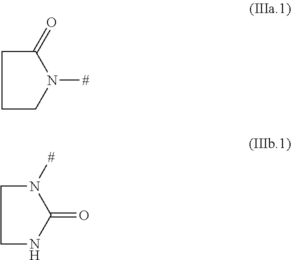

Preferred monomers f) are the compounds of the formula:

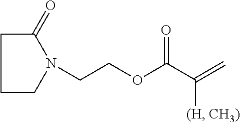

Suitable monomers f) having urea groups are, for example, N-vinyl- or N-allylurea or derivatives of imidazolidin-2-one. These include N-vinyl- and N-allylimidazolidin-2-one, N-vinyloxyethylimidazolidin-2-one, N-(2-(meth)acrylamidoethyl)imidazolidin-2-one, N-(2-(meth)acryloxyethyl)imidazolidin-2-one (=2-ureido(meth)acrylate), N-[2-((meth)acryloxyacetamido)ethyl]imidazolidin-2-one etc.

Preferred monomers f) having urea groups are N-(2-acryloxyethyl)imidazolidin-2-one and N-(2-methacryloxyethyl)imidazolidin-2-one. Particular preference is given to N-(2-methacryloxyethyl)imidazolidin-2-one (2-ureidomethacrylate, UMA).

Preferably, the component f) is used in an amount of from 0.1 to 20% by weight, particularly preferably from 0.5 to 10% by weight, based on the total weight of the monomers used for the polymerization.

### Monomer g)

In a further embodiment, the monomer composition used for the preparation of the copolymer composition can additionally comprise, in copolymerized form, at least one further monomer g) which is selected from esters of α,β-ethylenically unsaturated mono- and dicarboxylic acids with C₁-C₇-alkanols, esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols which have a primary or secondary amino group, and mixtures thereof.

Suitable esters of α,β-ethylenically unsaturated mono- and dicarboxylic acids with C₁-C₇-alkanols are, for example, methyl(meth)acrylate, methyl ethacrylate, ethyl(meth)acrylate, ethyl ethacrylate, n-butyl(meth)acrylate, tert-butyl(meth)acrylate, tert-butyl ethacrylate, n-pentyl(meth)acrylate, n-hexyl(meth)acrylate, n-heptyl(meth)acrylate, etc. Preferred monomers f) are the esters of α,β-ethylenically unsaturated mono- and dicarboxylic acids with C₁-C₃-alkanols, in particular methyl methacrylate.

Suitable additional monomers g) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable additional monomers g) are also 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

The component g) is preferably used in an amount of from 0.1 to 25% by weight, particularly preferably 0.5 to 20% by weight, based on the total weight of the monomers used for the polymerization.

### Monomer h)

At least one monomer h), different from the components a) to g) and copolymerizable therewith, can additionally be used for the preparation of the copolymer composition A) according to the invention.

Suitable compounds h) are selected from compounds of the general formulae IV a), IV b), IV c), IV d) and IV e)

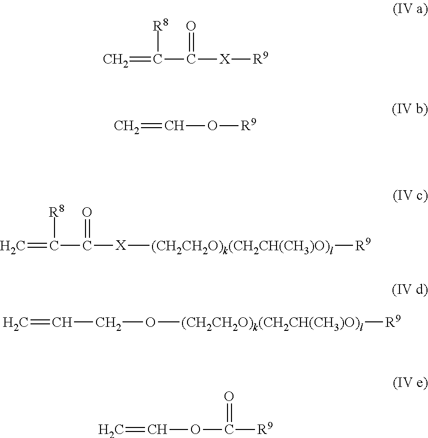

in which
the order of the alkylene oxide units is arbitrary,
k and l, independently of one another, are an integer from 0 to 1000, where the sum of k and l is at least 5,
R⁸ in the compounds (IVa) is hydrogen or C₁-C₄-alkyl, preferably methyl, and R⁸ in the compounds (IVc) is hydrogen or C₁-C₈-alkyl,
R⁹ in the compounds (IVa), (IVb) and (IVe) is C₈-C₃₀-alkyl or C₈-C₃₀-alkenyl, and R⁹ in the compounds (IVc) and (IVd) is hydrogen, C₁-C₃₀-alkyl, C₂-C₃₀-alkenyl or C₅-C₈-cycloalkyl,
X is O or a group of the formula NR¹⁰, in which R¹⁰ is H, alkyl, alkenyl, cycloalkyl, heterocycloalkyl, aryl or hetaryl.

Suitable monomers of the formula IV a) in which X is O are, for example, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable monomers of the formula IV a) in which X is NR¹⁰ are, for example, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleinyl(meth)acrylamide, oleyl(meth)acrylamide, linolyl(meth)acrylamide, linolenyl(meth)acrylamide, stearyl(meth)acrylamide, lauryl(meth)acrylamide, N-methyl-N-(n-octyl)(meth)acrylamide, N,N′-di(n-octyl)(meth)acrylamide and mixtures thereof.

Suitable monomers of the formula IV b) are, for example, n-octyl vinyl ether, 1,1,3,3-tetramethylbutyl vinyl ether, ethylhexyl vinyl ether, n-nonyl vinyl ether, n-decyl vinyl ether, n-undecyl vinyl ether, tridecyl vinyl ether, myristyl vinyl ether, pentadecyl vinyl ether, palmityl vinyl ether, heptadecyl vinyl ether, octadecyl vinyl ether, nonadecyl vinyl ether, arrachinyl vinyl ether, behenyl vinyl ether, lignocerenyl vinyl ether, cerotinyl vinyl ether, melissinyl vinyl ether, palmitoleinyl vinyl ether, oleyl vinyl ether, linolyl vinyl ether, linolenyl vinyl ether, stearyl vinyl ether, lauryl vinyl ether and mixtures thereof.

In the formulae IV c) and IV d), k is preferably an integer from 1 to 500, in particular 3 to 250. Preferably, l is an integer from 0 to 100.

Preferably, R⁸ in the formula IV c) is hydrogen, methyl, ethyl, n-propyl, isopropyl, n-butyl, sec-butyl, tert-butyl, n-pentyl or n-hexyl, in particular hydrogen, methyl or ethyl.

R⁹ in the formulae IV c) and IV d) is preferably hydrogen, methyl, ethyl, n-propyl, isopropyl, n-butyl, sec-butyl, n-pentyl, n-hexyl, n-heptyl, n-octyl, 1,1,3,3-tetramethylbutyl, ethylhexyl, n-nonyl, n-decyl, n-undecyl, tridecyl, myristyl, pentadecyl, palmityl, heptadecyl, octadecyl, nonadecyl, arrachinyl, behenyl, lignocerenyl, cerotinyl, melissinyl, palmitoleinyl, oleyl, linolyl, linolenyl, stearyl, lauryl.

Preferably, X in the formula IV c) is O or NH.

Suitable polyether acrylates IV c) are, for example, the polycondensation products of the aforementioned α,β-ethylenically unsaturated mono- and/or dicarboxylic acids and their acid chlorides, amides and anhydrides with polyetherols. Suitable polyetherols can be prepared easily by reacting ethylene oxide, 1,2-propylene oxide and/or epichlorohydrin with a starter molecule, such as water or a short-chain alcohol R⁹—OH. The alkylene oxides can be used individually, alternately one after the other or as a mixture. The polyether acrylates IV c) can be used on their own or in mixtures for the preparation of the polymers used according to the invention.

Suitable allyl alcohol alkoxylates IV d) are, for example, the etherification products of allyl chloride with corresponding polyetherols. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The allyl alcohol alkoxylates IV d) can be used on their own or in mixtures for the preparation of the polymers used according to the invention.

Suitable monomers IV e) are C₈-C₃₀-, preferably C₈-C₂₂-carboxylic acid vinyl esters. These include, for example, n-octyl vinyl ester, 1,1,3,3-tetramethylbutyl vinyl ester, ethylhexyl vinyl ester, n-nonyl vinyl ester, n-decyl vinyl ester, n-undecyl vinyl ester, tridecyl vinyl ester, myristyl vinyl ester, pentadecyl vinyl ester, palmityl vinyl ester, heptadecyl vinyl ester, octadecyl vinyl ester, nonadecyl vinyl ester, arrachinyl vinyl ester, behenyl vinyl ester, lignocerenyl vinyl ester, cerotinyl vinyl ester, melissinyl vinyl ester, palmitoleinyl vinyl ester, oleyl vinyl ester, linolyl vinyl ester, linolenyl vinyl ester, stearyl vinyl ester, lauryl vinyl ester and mixtures thereof.

Instead of or in addition to the aforementioned compounds h) of the general formulae IV a), IV b), IV c), IV d) and IV e), for the preparation of the copolymer composition A), additionally at least one compound h) can be used which is selected from urethane (meth)acrylates having alkylene oxide groups, esters of vinyl alcohol and allyl alcohol with C₁-C₇-monocarboxylic acids, C₁-C₇-alkyl vinyl ethers, vinylaromatics, vinyl halides, vinylidene halides, C₂-C₈-monoolefins, nonaromatic hydrocarbon radicals having at least two conjugated double bonds and mixtures thereof.

Suitable urethane (meth)acrylates having alkylene oxide groups h) are described in DE 198 38 851 (component e2)), to which reference is made here in its entirety.

Suitable additional monomers h) are also vinyl acetate, vinyl propionate, vinyl butyrate and mixtures thereof.

Suitable additional monomers h) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The aforementioned monomers h) can in each case be used individually or in the form of any desired mixtures.

Preferably, the component h) is used in an amount of from 0 to 20% by weight, based on the total weight of the monomers used for the polymerization. A suitable use amount for additional monomers h) is in a range from 0.1 to 10% by weight, in particular 0.2 to 5% by weight, based on the total weight of the compounds used for the polymerization.

In a first preferred embodiment of the process according to the invention, for the preparation of the copolymer composition A), based on the total weight of the monomers used for the polymerization,
98 to 99.9% by weight of acrylic acid a), and
0.1 to 2% by weight of at least one crosslinking compound c),
   are used.

In a further preferred embodiment of the process according to the invention, for the preparation of the copolymer composition A), based on the total weight of the monomers used for the polymerization,
93 to 99.7% by weight of acrylic acid a),
0.2 to 5% by weight of at least one compound d) with a free-radically polymerizable, α,β-ethylenically unsaturated double bond and at least one cationogenic and/or cationic group per molecule, preferably vinylimidazole, and
0.1 to 2% by weight of at least one crosslinking compound c),
   are used.

In a further preferred embodiment of the process according to the invention, for the preparation of the copolymer composition A), based on the total weight of the monomers used for the polymerization,
40 to 99.4% by weight of acrylic acid a),
0 to 40% by weight of at least one compound b), different from acrylic acid, having a free-radically polymerizable, α,β-ethylenically unsaturated double bond and at least one anionogenic and/or anionic group per molecule,
0.5 to 20% by weight of at least one compound g) and/or h), and
0.1 to 2% by weight of at least one crosslinking compound c),
   are used, with the proviso that the total amount of the monomers a) and b) is 78 to 99.4% by weight.

In a specific version to the aforementioned embodiment, for the preparation of the copolymer composition A), based on the total weight of the monomers used for the polymerization,
40 to 99.4% by weight of acrylic acid a),
0 to 40% by weight of at least one compound b), different from acrylic acid, having a free-radically polymerizable α,β-ethylenically unsaturated double bond and at least one anionogenic and/or anionic group per molecule,
0 to 20% by weight of at least one monomer g) which is selected from C₁-C₇-alkyl(meth)acrylates, in particular methyl(meth)acrylate, ethyl(meth)acrylate, n-butyl(meth)acrylate and mixtures thereof,
0 to 20% by weight of at least one compound h) which is preferably selected from C₈-C₂₂-alkyl(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof, and
0.1 to 2% by weight of at least one crosslinking compound c),
   are used, with the proviso that the total amount of the monomers a) and b) is 78 to 99.4% by weight and the sum of monomers g) and h) is 0.5 to 20% by weight.

In the two aforementioned embodiments, preferably methacrylic acid is used as component b). A preferred ester of an α,β-ethylenically unsaturated monocarboxylic acid with a C₁-C₇-alkanol is methyl methacrylate. A mixture of a C₁₈₋₂₂-alkyl polyethylene glycol methacrylate with methyl methacrylate is commercially available under the name Plex-6877-0. A mixture of a C₁₆₋₁₈-alkyl polyethylene glycol methacrylate with methacrylic acid is commercially available under the name Lutencryl AT 250.

In a further preferred embodiment of the process according to the invention, for the preparation of the copolymer composition A), based on the total weight of the monomers used for the polymerization,
48 to 99.4% by weight of acrylic acid a),
0 to 40% by weight of at least one compound b), different from acrylic acid, having a free-radically polymerizable α,β-ethylenically unsaturated double bond and at least one anionogenic and/or anionic group per molecule,
0.5 to 10% by weight, based on the total weight of a) and e), of at least one compound e) which is preferably selected from esters of α,β-ethylenically unsaturated mono- and dicarboxylic acids with C₈-C₃₀-alkanols, and
0.1 to 2% by weight of at least one crosslinking compound c),
   are used, with the proviso that the total amount of the monomers a) and b) is 88 to 99.4% by weight.

In the aforementioned embodiment, methacrylic acid is preferably used as component b).

In a further preferred embodiment of the process according to the invention, for the preparation of the copolymer composition A), based on the total weight of the monomers used for the polymerization,
40 to 99.4% by weight of acrylic acid a),
0 to 40% by weight of at least one compound b), different from acrylic acid, having a free-radically polymerizable α,β-ethylenically unsaturated double bond and at least one anionogenic and/or anionic group per molecule,
0 to 5% by weight of at least one compound d) with a free-radically polymerizable, α,β-ethylenically unsaturated double bond and at least one cationogenic and/or cationic group per molecule, preferably vinylimidazole,
0.1 to 30% by weight of at least one amide-group-containing compound e), preferably vinylpyrrolidone and/or vinylcaprolactam, and
0.1 to 2% by weight of at least one crosslinking compound c),
   are used, with the proviso that the total amount of the monomers a) and b) is 65 to 99.8% by weight.

In a specific version to the aforementioned embodiment, for the preparation of the copolymer composition A), based on the total weight of the monomers used for the polymerization,
65 to 98.7% by weight of acrylic acid a),
0.2 to 5% by weight of vinylimidazole,
1 to 30% by weight of vinylpyrrolidone and/or vinylcaprolactam,
0.1 to 2% by weight of at least one crosslinking compound c),
   are used.

### Glycerol Monostearate H1)

The component H1) is preferably used in an amount of from 0.1 to 15 parts by weight, particularly preferably 0.3 to 10 parts by weight, based on 100 parts by weight of the monomers used for the polymerization.

### Auxiliary H2)

Preferably, the component H2) is used in an amount of from 0.1 to 15 parts by weight, particularly preferably 0.3 to 10 parts by weight, based on 100 parts by weight of the monomers used for the polymerization.

Nonionic emulsifiers suitable as auxiliary H2) are preferably selected from
alkoxylated primary C₈-C₃₀-alcohols,
esters of C₈-C₃₀-carboxylic acids with polyhydric alcohols and alkoxylates thereof,
alkoxylated fatty acid alkyl esters,
amine oxides,
fatty acid alkanolamides,
polyhydroxy fatty acid amides,
mixtures of the aforementioned emulsifiers,
   with the proviso that they have an HLB value in the range from 4 to 10.

Alkoxylated primary alcohols suitable as H2) have preferably 10 to 22 carbon atoms, particularly preferably 12 to 20 carbon atoms. They are alkoxylated preferably with 1 to 50, particularly preferably 1 to 30, such as, for example, 2 to 20, mol of alkylene oxide per mole of alcohol. For the alkoxylation it is possible to use, for example, ethylene oxide, propylene oxide, 1,2-butylene oxide and mixtures thereof. Preference is given to ethylene oxide (EO). They are preferably alcohols with linear or branched alkyl or alkenyl radicals, where the latter may also have two or more nonadjacent double bonds. Preference is given to alcohols in which the alcohol radical is linear or methyl-branched in the 2 position, and/or alcohol mixtures with linear and methyl-branched radicals, as are usually present in oxo alcohol radicals. Particular preference is given to alkoxylates, specifically ethoxylates with alcohols of natural origin, and also oxo alcohol alkoxylates and Guerbet alcohol alkoxylates. These include, in particular, alkoxylates of alcohols and alcohol mixtures having 8 to 30, preferably 10 to 22, carbon atoms and obtainable by reducing natural fatty acids, for example of n-decanol, lauric alcohol, myristic alcohol, cetyl alcohol, stearic alcohol, oleic alcohol, lignoceryl alcohol, ceryl alcohol, etc. The stated degrees of alkoxylation are in each case statistical average values which may be an integer or a fraction for a specific product. Also suitable are alkoxylated alcohols which have a narrowed homolog distribution (narrow range ethoxylates, NRE). It is also possible to use alkoxylated alcohols which contain EO and PO groups together in the molecule. In this connection, it is possible to use block copolymers with EO-PO block units or PO-EO block units, but also EO-PO-EO copolymers or PO-EO-PO copolymers. It is of course also possible to use as H2) mixed alkoxylated primary alcohols in which EO and PO units are not distributed blockwise, but randomly. Such products are obtainable, for example, through the simultaneous action of ethylene oxide and propylene oxide on primary alcohols.

Esters of C₈-C₃₀-carboxylic acids with polyhydric alcohols suitable as auxiliary H2) are preferably derived from linear or branched, saturated or mono- or polyunsaturated carboxylic acids having 8 to 30 carbon atoms, preferably 10 to 22 carbon atoms, in particular 10 to 18 carbon atoms, in the alkyl or alkenyl radical, such as caprylic acid, pelargonic acid, undecanoic acid, lauric acid, tridecanoic acid, myristic acid, pentadecanoic acid, palmitic acid, margaric acid, stearic acid, isostearic acid, nonadecanoic acid, arachic acid, behenic acid, palmitoleic acid, oleic acid, linoleic acid, linolenic acid, arachidonic acid and mixtures thereof. Preference is given to lauric acid, stearic acid, isostearic acid, palmitic acid and oleic acid.

Suitable polyhydric alcohols are preferably selected from straight-chain, branched or carbocyclic, saturated or unsaturated hydrocarbon compounds having at least 3 carbon atoms and at least 3 (esterifiable) hydroxyl groups. Unsaturated hydrocarbon compounds here can have 1 or more, preferably 1, 2 or 3, C—C double bonds. Mixtures of such polyols can likewise be used. The polyol is in particular a straight-chain or branched saturated hydrocarbon having 3 to 30 carbon atoms and 3 to 10 hydroxyl groups. Preferred examples of polyols which can be used are: glycerol, di-, tri- and polyglycerols, low molecular weight partially or completely hydrolyzed polyvinyl acetate, 1,2,4-butanetriol, trimethylolmethane, trimethylolethane, trimethylolpropane, trimethylolbutane, 2,2,4-trimethyl-1,3-pentanediol, pentaerythritol, ditrimethylolpropane, dipentaerythritol, tripentaerythritol, D-, L- and meso-erythritol, D- and L-arabitol, adonitol, xylitol, sorbitol, mannitol, dulcitol, inositols and mixtures thereof.

Also suitable as auxiliary H2) are alkoxylates of esters of C₈-C₃₀-carboxylic acids with polyhydric alcohols. The alkoxylation can take place with ethylene oxide, propylene oxide and/or butylene oxide. Block copolymers or random copolymers may be present. Per mole of alcohol, they usually comprise 1 to 50 mol, preferably 2 to 30 mol, of at least one alkylene oxide. A preferred alkylene oxide is ethylene oxide.

A further class of preferably used nonionic surfactants which can be used as auxiliary H2) are alkoxylated, preferably ethoxylated or ethoxylated and propoxylated fatty acid alkyl esters, preferably having 1 to 4 carbon atoms in the alkyl chain, in particular fatty acid methyl esters, as are described, for example, in the Japanese patent application JP 58/217598 or which are preferably prepared by the process described in the international patent application WO-A-90/13533.

Also suitable as auxiliaries H2) are nonionic surfactants of the amine oxide type, for example N-cocoalkyl-N,N-dimethylamine oxide and N-tallow-alkyl-N,N-dihydroxyethylamine oxide, and also fatty acid alkanolamides. The amount of these nonionic surfactants is preferably not more than that of the ethoxylated fatty alcohols, in particular not more than half thereof.

Further suitable auxiliaries H2) are polyhydroxy fatty acid amides of the formula (2),

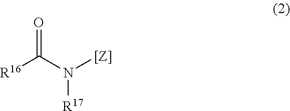

in which R¹⁶C(═O) is an aliphatic acyl radical having 6 to 22 carbon atoms, R¹⁷ is hydrogen, an alkyl or hydroxyalkyl radical having 1 to 4 carbon atoms and [Z] is a linear or branched polyhydroxyalkyl radical having 3 to 10 carbon atoms and 3 to 10 hydroxyl groups. The polyhydroxy fatty acid amides are known substances which can usually be obtained by reductive amination of a reducing sugar with ammonia, an alkylamine or an alkanolamine and subsequent acylation with a fatty acid, a fatty acid alkyl ester or a fatty acid chloride.

The group of polyhydroxy fatty acid amides also includes compounds of the formula (3)

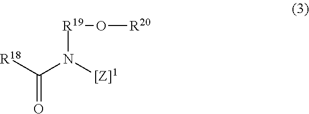

in which R¹⁸ is a linear or branched alkyl or alkenyl radical having 7 to 12 carbon atoms, R¹⁹ is a linear, branched or cyclic alkylene radical having 2 to 8 carbon atoms or an arylene radical having 6 to 8 carbon atoms and R²⁰ is a linear, branched or cyclic alkyl radical or an aryl radical or an oxy-alkyl radical having 1 to 8 carbon atoms, where C₁-C₄-alkyl or phenyl radicals are preferred, and [Z]¹ is a linear polyhydroxyalkyl radical whose alkyl chain is substituted with at least two hydroxyl groups, or alkoxylated, preferably ethoxylated or propoxylated derivatives of this radical. [Z]¹ is preferably obtained by reductive amination of a sugar, for example glucose, fructose, maltose, lactose, galactose, mannose or xylose. The N-alkoxy- or N-aryloxy-substituted compounds can then be converted to the desired polyhydroxy fatty acid amides, for example in accordance with WO-A-95/07331, by reaction with fatty acid methyl esters in the presence of an alkoxide as catalyst.

Nonionic emulsifiers suitable as auxiliary H2) are specifically (HLB values and optionally trade name in brackets):

Polyoxyethylene sorbitol beeswax derivative (4.0; Atlas G-1727),
Propylene glycol fatty acid ester (4.1; Emcol PM-50),
Sorbitan monooleate (4.3; Span 80),
Sorbitan monooleate (4.3; Arlacel 80),
Propylene glycol monolaurate (4.5; Atlas G-917),
Propylene glycol monolaurate (4.5; Atlas G-3851),
Propylene glycol fatty acid ester (4.5; Emcol PL-50),
Sorbitan monostearate (4.7; Span 60 or Arlacel 60),
Diethylene glycol monooleate (4.7; Atlas G-2139),
Diethylene glycol fatty acid ester (4.7; Emcol DO-50),
Diethylene glycol monostearate (4.7; Atlas G-2146),
Diethylene glycol fatty acid ester (4.7; Emcol DS-50),
Polyoxyethylene sorbitol beeswax derivative (5.0; Atlas G-1702),
Diethylene glycol fatty acid ester (5.1; Emcol DP-50),
Diethylene glycol fatty acid ester (5.6; Emcol DM-50),
Polyoxyethylene sorbitol beeswax derivative (6.0; Atlas G-1725),
Diethylene glycol monolaurate (6.1; Atlas G-2124),
Diethylene glycol fatty acid ester (6.1; Emcol DL-50),
Diethylene glycol monolaurate (6.5; Glaurin),
Sorbitan monopalmitate (6.7; Span 40 or Arlacel 40),
Polyoxyethylene dioleate (7.5; Atlas G-2242),
Tetraethylene glycol monostearate (7.7; Atlas G-2147),
Tetraethylene glycol monooleate (7.7; Atlas G-2140),
Polyoxypropylene mannitol dioleate (8.0; Atlas G-2800),
Polyoxyethylene sorbitol lanolin oleate derivative (8.0; Atlas G-1493),
Polyoxyethylene sorbitol lanolin derivative (8.0; Atlas G-1425),
Polyoxypropylene stearate (8.0; Atlas G-3608),
Sorbitan monolaurate (8.6; Span 20 or Arlacel 20),
Polyoxyethylene fatty acid (9.0; Emulphor VN-430),
Polyoxyethylene sorbitol beeswax derivative (9.0; Atlas G-1734),
Poly(oxyethylene-oxypropylene) oleate (9.0; Atlas G-2111),
Tetraethylene glycol monolaurate (9.4; Atlas G-2125),
Polyoxyethylene lauryl ether (9.5; Brij 30),
Polyoxyethylene sorbitan monostearate (9.6; Tween 61),
Hexaethylene glycol monostearate (9.6; Atlas G-2154),
Polyoxyethylene sorbitan monooleate (10.0; Tween 81),
and mixtures thereof.

Nonionic emulsifiers particularly suitable as auxiliary H2) are the Dehymuls® grades from COGNIS Care Chemicals. These include:
Dehymuls HRE 7 (hydrogenated castor oil alkoxylated with 7 mol of ethylene oxide),
Dehymuls LE (PEG-30 dipolyhydroxystearate),
Dehymuls E (mixture of higher molecular weight fatty acid esters, fatty acid salts and oil-binding additives; dicocoyl pentaerythrityl distearyl citrate and sorbitan sesquioleate and beeswax (cera alba) and aluminum stearate),
Dehymuls K (petrolatum and decyl oleate and dicocoyl pentaerythrityl distearyl citrate and sorbitan sesquioleate and microcrystalline wax (cera microcrystallina) and mineral oil and beeswax (cera alba) and aluminum stearate),
Dehymuls PGPH (polyglycerol poly-12-hydroxystearic acid ester),
Dehymuls SML (sorbitan monolaurate),
Dehymuls SMO (sorbitan monooleate),
Dehymuls SMS (sorbitan monostearate),
Dehymuls SSO (sorbitan sesquioleate),
   and mixtures thereof.

Nonionic emulsifiers particularly suitable as auxiliary H2) are also the Hypermer® grades from ICI. These include:
Hypermer LP6 (polymeric fatty acid ester, molecular weight (MW) about 4300)
Hypermer LP7
Hypermer B239 (block copolymer of a polyhydroxy fatty acid and polyethylene oxide, MW ca. 3500),
Hypermer B246 (block copolymer of a polyhydroxy fatty acid and polyethylene oxide, MW ca. 7500)
Hypermer B261 (block copolymer of a polyhydroxy fatty acid and polyethylene oxide, MW ca. 9600)
Hypermer 2234
Hypermer E-464 (copolymer with a long hydrophobic alkylene chain and various anionic/nonionic hydrophiles, MW ca. 2300)
Hypermer IL2296
Hypermer A-109 (block copolymer of a fatty acid or long-chain alkylene and EO),
Hypermer A-409 (block copolymer of a fatty acid or long-chain alkylene and EO).

Water-insoluble natural waxes particularly suitable as auxiliary H2) are beeswax, berry wax, rice germ oil wax, candelilla wax, carnauba wax, Japan wax, esparto grass wax, cork wax, guaruma wax, sugarcane wax, ouricury wax, montan wax, jojoba wax, shea butter, shellac wax, spermaceti, lanolin (wool wax), uropygial grease, ceresin and ozokerite (earth wax).

The component H2) particularly preferably comprises a beeswax or consists of a beeswax.

Beeswax is a mixture of about 70-75% by weight of various esters of C₂₆-C₃₂-alcohols, primarily of palmitic acid, hydroxypalmitic acid, α,β-dihydroxypalmitic acid and cerotic acid, ca. 14% free wax acid, ca. 12% hydrocarbons, ca. 1% fatty acid esters, and free wax alcohols with at most 1%. Suitable beeswaxes are commercially available. These include, for example, the following available from Kahl GmbH & Co. KG, D-22946 Trittau:

| | | MP/DP | | |
|---|---|---|---|---|
| Product | Wax | [° C.] | INCI name | CAS No. |
| 8104 | | | | |
| 8139 Bio | Beeswax | 61-66 | Cera alba | 8012-89-3 |
| beeswax | organic | | beeswax | |
| Cera Flava | Beeswax | 61-66 | | 8012-89-3 |
| | yellow | | | |
| Cera alba | Beeswax white | 62-65 | | 8012-89-3 |
| 8070 white | Beeswax | 62-65 | | 8012-89-3 |
| | bleached | | | |
| 1540 | Beeswax | 61-65 | | |
| | synthetic | | | |
| 6103 | Beeswax | 61-65 | | |
| | synthetic | | | |
| | animalfree | | | |

The sum of the components H1) and H2) is preferably used in an amount of from 0.2 to 30 parts by weight, particularly preferably 0.6 to 20 parts by weight, based on 100 parts by weight of the monomers used for the polymerization.

### Additional Auxiliary H3)

In a specific embodiment of the process according to the invention, for the preparation of the copolymer composition A), additionally at least one further auxiliary H3) is used which is selected from nonionic emulsifiers with an HLB value in the range from greater than 10 to 16. The HLB value of the component H3) is preferably in a range from 12 to 14.

The auxiliary H3) is preferably water-soluble.

Preferably, the component H3), if present, is used in an amount of from 0.1 to 15 parts by weight, particularly preferably 0.3 to 10 parts by weight, based on 100 parts by weight of the monomers used for the polymerization. Preferably, the amount of the sum of the components H1) and H2) is greater than or equal to the amount of H3).

Nonionic emulsifiers suitable as auxiliary H3) are preferably selected from
alkoxylated primary C₈-C₃₀-alcohols,
esters of C₈-C₃₀-carboxylic acids with polyhydric alcohols and alkoxylates thereof,
alkoxylated fatty acid alkyl esters,
amine oxides,
fatty acid alkanolamides,
polyhydroxy fatty acid amides,
mixtures of the aforementioned emulsifiers,
   with the proviso that they have an HLB value in the range from greater than 10 to 16.

Nonionic emulsifiers suitable as auxiliary H3) are specifically (HLB values and optionally trade name in brackets):

Polyoxyethylene ester of mixed fatty acids and resin acids (10.2; Atlas G-1218),
Polyoxyethylene cetyl ether (10.3; Atlas G-3806),
Polyoxyethylene sorbitan tristearate (10.5; Tween 65),
Polyoxyethylene lauryl ether (10.8; Atlas G-3705),
Polyoxyethylene sorbitan trioleate (11; Tween 85),
Poly(oxyethylene-oxypropylene) oleate (11; Atlas G-2116),
Polyoxyethylene lanolin derivative (11; Atlas G-1790),
Polyoxyethylene monooleate (11.1; Atlas G-2142),
Polyoxyethylene monostearate (11.1; Myrj 45),
Polyoxyethylene monooleate (11.4; Atlas G-2141),
Polyethylene glycol(400) monooleate (11.4),
Polyoxyethylene monopalmitate (11.6; Atlas G-2076),
Polyoxyethylene monostearate (11.6; S-541),
Polyoxyethylene monolaurate (12.8; Atlas G-2127),
Polyoxyethylene alkylphenol (12.8; Igepal CA-630),
Polyoxyethylene sorbitol lanolin derivative (13; Atlas G-1431),
Polyoxyethylene alkylaryl ether (13; Atlas G-1690),
C₁₀-Guerbet alcohol alkoxylate(7 EO) (13; Lutensol® XL 70),
C₁₀-Guerbet alcohol ethoxylate(7 EO) (13; Lutensol XP 70),
Polyoxyethylene monolaurate (13.1; S-307),
Polyethylene glycol(400) monolaurate (13.1),
Polyoxyethylene lauryl ether (13.1; Atlas G-2133),
Polyoxyethylene castor oil (13.3; Atlas G-1794),
Polyoxyethylene vegetable oil (13.3; Emulphor EL-719),
Polyoxyethylene sorbitan monolaurate (13.3; Tween 21),
Polyoxyethylene esters of mixed fatty acids and resin acids (13.5; Renex 20),
Polyoxyethylene sorbitol lanolin derivative (14; Atlas G-1441),
Polyoxyethylene sorbitan monolaurate (14.9; Atlas G-7596J),
Polyoxyethylene sorbitan monostearate (14.9; Tween 60),
Polyoxyethylene sorbitan monooleate (15; Tween 80),
Polyoxyethylene monostearate (15.0; Myrj 49),
Polyoxyethylene monooleate (15.1; Atlas G-2144),
Polyoxyethylene oleyl ether (15.3; Atlas G-3915),
Polyoxyethylene stearyl alcohol (15.3; Atlas G-3720),
Polyoxyethylene oleyl alcohol (15.4; Atlas G-3920),
Polyoxyethylene fatty alcohol (15.4; Emulphor ON-870),
Polyoxyethylene glycol monopalmitate (15.5; Atlas G-2079),
Polyoxyethylene sorbitan monopalmitate (15.6; Tween 40),
Polyoxyethylene cetyl alcohol (15.7; Atlas G-3820),
Poly(oxyethylene-oxypropylene) stearate (15.7; Atlas G-2162),
Polyoxyethylene sorbitol lanolin derivative (16.0; Atlas G-1471),
Polyoxyethylene monostearate (16.0; Myrj 51).

Preferably, the component H3) comprises at least one poly(oxyethylene) sorbitan monolaurate, e.g. a poly(oxyethylene) sorbitan monolaurate with 20 ethylene oxide units which is commercially available under the trade name Tween® 20.

### Precipitation Polymerization

According to the invention, the preparation of the copolymer composition A) takes place by the method of precipitation polymerization. For this polymerization, solvents are used in which the starting materials for the polymerization are soluble and the polymer which is formed is insoluble. Preference is given to using an anhydrous aprotic solvent or solvent mixture. Suitable solvents are, for example, aromatic hydrocarbons such as toluene, xylenes, benzene; aliphatic and cycloaliphatic hydrocarbons such as n-alkanes or cyclohexane; esters of acetic acid such as ethyl acetate or butyl acetate; ethers, such as, for example, diethyl ether, dipropyl ether, dibutyl ether, methyl tert-butyl ether or diethylene glycol dimethyl ether; ketones such as acetone or methyl ethyl ketone, and mixtures of these solvents.

Preferably, the polymerization takes place in a mixture of cyclohexane and ethyl acetate. The ratio of cyclohexane to ethyl acetate is preferably in a range from 60:40 to 30:70.

The precipitation polymerization is usually carried out at temperatures of from 20 to 150° C., preferably 40 to 120° C., in particular 60 to 100° C.

The precipitation polymerization is usually carried out at pressures of from 1 to 15 bar, in particular 1 to 6 bar. If the polymerization is not carried out under increased pressure, the solvent or solvent mixture determines the maximum reaction temperature through the corresponding boiling temperatures.

To prepare the polymers, the monomers can be polymerized with the help of initiators which form free radicals.

Initiators for the free-radical polymerization which can be used are the peroxo and/or azo compounds customary for this purpose, for example alkali metal or ammonium peroxidisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-toluoyl) peroxide, didecanoyl peroxide, dioctanoyl peroxide, tert-butyl peroctoate, dilauroyl peroxide, tert-butyl perisobutyrate, tert-butyl peracetate, di-tert-amyl peroxide, tert-butyl hydroperoxide, 2,2′-azobisisobutyronitrile, azobis(2-amidinopropane) dihydrochloride, azobis(2,4-dimethylvaleronitrile) or 2,2′-azobis(2-methylbutyronitrile).

Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{I}.

In a special embodiment, for the preparation of the copolymers according to the invention, at least two free-radical initiators are used which permit an essentially independent initiation in at least two phases. Here, copolymers with particularly low residual monomer contents can be achieved. For the copolymerization, preference is then given to using at least two initiators whose disintegration temperatures differ from one another by at least 10° C. Within the context of the invention, the disintegration temperature is defined as the temperature at which 50% of the molecules disintegrate into free radicals within 2.5 hours. Preferably, the copolymerization takes place in the case of this procedure until conclusion of the precipitation of the copolymer at a temperature greater than or equal to the lower disintegration temperature and less than the higher disintegration temperature, and after the precipitation, a further reaction takes place at a temperature greater than or equal to the higher disintegration temperature.

The process according to the invention preferably comprises a first polymerization phase at a first polymerization temperature and a second polymerization phase at a second polymerization temperature above the first polymerization temperature, where, for the polymerization, at least two initiators are used whose half-lives at the first polymerization temperature differ in such a way that at least one of these initiators disintegrates into free radicals during the first polymerization phase and at least one of these initiators essentially does not disintegrate into free radicals during the first polymerization phase and disintegrates into free radicals during the second polymerization phase. Preferably, in the case of this procedure, the second polymerization phase starts essentially after precipitation of the copolymer. “Essentially” after precipitation of the copolymer is understood as meaning that the copolymer is present in precipitated form preferably to at least 80% by weight, preferably at least 90% by weight, in particular at least 95% by weight, based on the total weight of the copolymer.

The half-life of an initiator can be determined by customary methods known to the person skilled in the art, as described, for example, in the publication “Initiators for high polymers”, Akzo Nobel, No. 10737. Preferably, the half-life of the first polymerization initiator at the first polymerization temperature and of the second polymerization initiator at the second polymerization temperature is in a range from about 1 minute to 3 hours, particularly preferably 5 minutes to 2.5 hours. If desired, it is also possible to use shorter half-lives, e.g. from 1 second to 1 minute or longer half-lives than 3 hours, provided it is ensured that the initiator(s) disintegrating at the higher temperature disintegrates into free radicals essentially during the second polymerization phase.

Preferably, the initiator system used comprises at least two initiators whose disintegration temperatures differ from one another by at least 15° C. The initiator which disintegrates at the lower temperature preferably has a disintegration temperature of from 50 to 100° C. The initiator disintegrating at the higher temperature preferably has a disintegration temperature of from 80 to 150° C.

In general, the precipitation polymerization can be carried out at solids contents up to ca. 25%. Preference is given to a range from 15 to 22%. By using the auxiliaries H1), H2) and optionally H3), it is generally possible to dispense with the use of further protective colloids. If desired, however, in the process according to the invention, a protective colloid different from H1), H2) and H3) can additionally be used. Of suitability are the known protective colloid polymers which readily dissolve in the solvents used and do not react with the monomers. Suitable polymers are, for example, copolymers of maleic acid with vinyl alkyl ethers and/or olefins having 8 to 20 carbon atoms or corresponding copolymers of maleic acid half-esters with C₁₀-C₂₀-alcohols or else mono- and diamides of maleic acid with C₁₀-C₂₀-alkylamines, and polyvinyl alcohol ethers with alkyl groups which carry 1 to 20 carbon atoms and also polyvinyl methyl, ethyl, isobutyl or octadecyl ethers. The amount of protective colloid polymer used is generally 0.05 to 4% by weight, preferably 0.1 to 2% by weight (based on the total weight of the monomers used).

The polymerization can be carried out by initially introducing some of the solvent, the auxiliaries H1) and H2) and optionally protective colloid polymer, heating, and carrying out the polymerization by adding initiator, monomer(s) and crosslinker (in each case possibly dissolved in the same solvent or solvent mixture).

In an alternative embodiment, the crosslinker c) can be initially introduced in part or completely. It is likewise possible to initially introduce some of the monomers and the initiator (e.g. up to 50%, preferably up to 35%). The initial charge can then be heated to polymerization temperature and, after the reaction has started, the remainder of the mixture to be polymerized can be added according to the progress of the polymerization.

It is likewise possible not to initially introduce the crosslinker c) used, but to add it completely in the course of the polymerization.

The auxiliaries H1) and/or H2) are preferably initially introduced at least partly prior to the start of the polymerization. H1) and H2) are particularly preferably initially introduced completely prior to the start of the polymerization.

If at least one auxiliary H3) is used, the addition preferably takes place after at least 90% of the monomers have reacted.

The precipitated polymer is then isolated from the reaction mixture, for which purpose any general method for isolating the polymers in the conventional precipitation polymerization can be used. Such methods are filtration, centrifugation, evaporation of the solvent or combinations of these methods.

The copolymer composition can if desired be subjected to a purification. This serves, for example, to remove nonpolymerized constituents and/or at least some of the auxiliaries. In one preferred embodiment, the copolymer composition A) is isolated after the precipitation polymerization and subjected to a washing with a liquid washing medium. Suitable washing media are in principle the same solvents as are suitable for the polymerization. However, for easier drying of the polymers, it is advisable to use solvents with a low boiling point, such as, for example, acetone.

To remove impurities, the copolymer composition A) can be subjected to a treatment with a washing medium once or several times in succession. For this, in a suitable device, the copolymer composition is brought into close contact with the washing medium and the washing medium is then separated off from the copolymer composition. Suitable devices are, for example, stirred reactors. In this connection, the treatment with the washing medium can take place in the container also used for the polymerization. The separation of copolymer and washing medium takes place, for example, by filtration or centrifugation. To increase the rate, the filtration can take place under increased pressure on the polymer side or reduced pressure on the discharge side.

### Copolymer Composition

The invention further provides the copolymer composition A) obtainable by the process described above.

In addition to the polymer particles obtained during the precipitation polymerization, the copolymer composition A) according to the invention can comprise at least one further component. This includes at least one of the auxiliaries H1) and/or H2). The auxiliaries H1) and/or H2) can, if desired, be removed partly or completely from the copolymer composition A), e.g. by at least one washing step, as described previously.

If, for the preparation of the copolymer composition A) according to the invention, at least one auxiliary H3) is used, then A) can comprise the auxiliary(s) H3) as further component. A special embodiment is therefore a copolymer composition A) which comprises at least one component H3). A) then comprises preferably at least one component H3) in an amount of from 0.01 to 15% by weight, particularly preferably 0.1 to 10% by weight, based on the total weight of the copolymer composition A). The auxiliary H3) can have an advantageous effect on the application-related properties of the copolymer composition A), e.g. through an increase in the dissolution rate or reduced dust formation.

The auxiliaries H1), H2) and/or H3) can also have an advantageous effect on one or more other application-related properties of the copolymer composition A), e.g. by helping products which are not very free-flowing, or controlling particle size, molecular weight, morphology, etc.

The auxiliaries H1), H2) and/or H3) can also have an advantageous effect on one or more other application-related properties of formulations of the copolymer composition A). Thus, for example, the presence of at least one of these auxiliaries may have an advantageous effect on the clarity of the gels formulated with A).

The copolymer compositions A) according to the invention and the copolymers present therein are characterized by their pH-dependent solubility.

The anionogenic groups (acid groups) of the copolymers present in the copolymer compositions A) can be partly or completely neutralized with a base. Bases which can be used for the neutralization of the polymers are alkali metal bases such as sodium hydroxide solution, potassium hydroxide solution, sodium carbonate, sodium hydrogencarbonate, potassium carbonate or potassium hydrogencarbonate, and alkaline earth metal bases such as calcium hydroxide, calcium oxide, magnesium hydroxide or magnesium carbonate, and also amines. Suitable amines are, for example, C₁-C₆-alkylamines, preferably n-propylamine and n-butylamine, dialkylamines, preferably diethylpropylamine or dipropylmethylamine, trialkylamines, preferably triethylamine and triisopropylamine. Preference is given to amino alcohols, e.g. trialkanolamines, such as triethanolamine, alkyldialkanolamines, such as methyl- or ethyldiethanolamine and dialkylalkanolamines, such as dimethylethanolamine and 2-amino-2-methyl-1-propanol. The neutralization of the acid groups can also be carried out with the help of mixtures of two or more bases. The neutralization can take place partly or completely depending on the intended use.

If the copolymers present in the copolymer composition A) are to be both quaternized and neutralized, then preferably the quaternization takes place first and then the neutralization.

The copolymers present in the copolymer composition A) are advantageously suitable for modifying the rheological properties of aqueous compositions. These may be, for example, an aqueous active ingredient or effect substance composition. These may quite generally be, for example, cosmetic compositions, pharmaceutical compositions, hygiene products, coatings, compositions for the paper industry and also the textile industry.

In one preferred embodiment, the compositions comprise at least one water-soluble or at least water-dispersible active ingredient or effect substance. The copolymers present in the copolymer composition A) are of course also suitable to be used for modifying the rheological properties of compositions which comprise at least one water-insoluble (hydrophobic) active ingredient or effect substance.

Within the context of the present invention, “modification of rheological properties” is to be understood in the broad sense. The copolymers present in the copolymer composition A) are generally suitable for thickening the consistency of aqueous compositions in a wide range. Depending on the basic consistency of the liquid compositions, flow properties from thin-liquid ranging to solid (in the sense “no longer flowable”) can generally be achieved, depending on the use amount of the copolymer. “Modification of rheological properties” is therefore understood as meaning, inter alia, the increase in the viscosity of liquids, the improvement in the thixotropic properties of gels, the solidification of gels and waxes etc. The compositions according to the invention are preferably suitable for the formulation of aqueous cosmetic and pharmaceutical products. Preferably, the compositions of the copolymers A) are generally clear. Consequently, formulations, in particular cosmetic formulations, can be advantageously colored without impairment from the intrinsic color of the compositions. Furthermore, the compositions can be formulated in the form of clear gels.

The copolymer compositions A) prepared in the presence of the auxiliary system according to the invention are characterized overall by advantageous rheological properties. The rheology-modifying properties can be further controlled via the type and use amount of the monomers used for the preparation of the copolymer compositions A). This applies especially to the type and amount of crosslinker c) used. This further applies especially for the use of surface-active monomers in the preparation of A), such as, for example, the polyether acrylates IV c) or allyl alcohol alkoxylates IV d). A 0.2% strength by weight aqueous solution of a copolymer composition A) generally has a viscosity in the range from 7000 to 20 000 mPas (values determined by means of Brookfield viscometer at 23° C. and 100 s⁻¹).

A 0.5% strength by weight aqueous solution of a copolymer composition A) generally has a viscosity in the range from 28 000 to 60 000 mPas (values determined by means of Brookfield viscometer at 23° C. and 100 s⁻¹).

The copolymer compositions A) are suitable both for the preparation of homogeneous-phase aqueous compositions, and also for the formulation of heterogeneous-phase compositions which additionally comprise at least one water-insoluble (hydrophobic) liquid or solid compound. “Homogeneous-phase compositions” have only a single phase irrespective of their number of constituents. “Heterogeneous-phase compositions” are disperse systems of two or more components that are immiscible with one another. These include solid/liquid, liquid/liquid and solid/liquid/liquid compositions, such as dispersions and emulsions, e.g. O/W and W/O formulations which have at least one of the oil and/or fat components described in more detail below and water as immiscible phases. In principle, the copolymers A) can be used either in the water phase or else in the oil phase. In general, heterogeneous-phase liquid/liquid compositions comprise the copolymers A) essentially in the water phase.

The copolymer compositions A) according to the invention are very generally suitable for the preparation of active ingredient or effect substance compositions comprising

A) at least one copolymer composition, as defined previously,
B) at least one active ingredient or effect substance and
C) optionally at least one further auxiliary different from A) and B).

Active ingredients for cosmetics (e.g. hair and skin cosmetics), medicaments, hygiene compositions, textile treatment compositions etc., i.e. substances which generally develop an effect even at low concentration, e.g. a cosmetic effect on skin and/or hair, a pharmacological effect in an organism, a cleaning and/or disinfecting effect, a modification of a textile substance, e.g. a crease-free finishing, and effect substances, which impart a certain property to living things or inanimate substrates, for example color pigments for make-up or emulsion paints, are often formulated and used in the form of aqueous active ingredient or effect substance compositions.

The active ingredient and effect substance compositions according to the invention comprise the polymer component A) preferably in an amount of from 0.01 to 50% by weight, particularly preferably 0.05 to 30% by weight, in particular 0.1 to 20% by weight, based on the total weight of the composition. Even in small use amounts, the copolymer compositions according to the invention advantageously exhibit good application-related properties, e.g. a good thickening effect. In a specific embodiment, the active ingredient and effect substance compositions according to the invention comprise polymer component A) in an amount of from 0.1 to 5% by weight, based on the total weight of the composition.

The components B) and C) are selected according to the desired field of use of the composition. Besides components which are typical for the field of use (e.g. certain pharmaceutical active ingredients), they are selected, for example, from carriers, excipients, emulsifiers, surfactants, preservatives, fragrances, thickeners different from component A), polymers, gel formers, dyes, pigments, photoprotective agents, consistency regulators, antioxidants, antifoams, antistats, resins, solvents, solubility promoters, neutralizing agents, stabilizers, sterilizing agents, propellants, drying agents, opacifiers, etc.

The compositions preferably have a carrier component C) which is selected from water, hydrophilic carriers different from water and mixtures thereof.

Suitable hydrophilic carriers C) are, for example, mono-, di- or polyhydric alcohols having preferably 1 to 8 carbon atoms, such as ethanol, n-propanol, isopropanol, propylene glycol, glycerol, sorbitol, etc.

The compositions according to the invention can comprise, as active ingredient, e.g. as cosmetic and/or pharmaceutical active ingredient B) (and also optionally as auxiliary C)), at least one polymer which differs from the copolymer compositions A) according to the invention. These include, quite generally, anionic, cationic, amphoteric and neutral polymers.

Examples of anionic polymers are copolymers of acrylic acid and acrylamide and salts thereof; sodium salts of polyhydroxycarboxylic acids, water-soluble or water-dispersible polyesters, polyurethanes, e.g. Luviset PUR® from BASF, and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally further vinyl esters (e.g. Luviset® grades), maleic anhydride copolymers, optionally reacted with alcohol, anionic polysiloxanes, e.g. carboxyfunctional ones, t-butyl acrylate, methacrylic acid (e.g. Luviskol® VBM), copolymers of acrylic acid and methacrylic acid with hydrophobic monomers, such as, for example, C₄-C₃₀-alkyl esters of (meth)acrylic acid, C₄-C₃₀-alkylvinyl esters, C₄-C₃₀-alkyl vinyl ethers and hyaluronic acid. One example of an anionic polymer is also the methyl methacrylate/methacrylic acid/acrylic acid/urethane acrylate copolymer available under the name Luviset® Shape (INCI name: Polyacrylate-22). Examples of anionic polymers are also vinyl acetate/crotonic acid copolymers, as are commercially available, for example, under the names Resyn®(National Starch) and Gafset® (GAF), and vinylpyrrolidone/vinyl acrylate copolymers available, for example, under the trade name Luviflex® (BASF). Further suitable polymers are the vinylpyrrolidone/acrylate terpolymer available under the name Luviflex® VBM-35 (BASF) and polyamides containing sodium sulfonate or polyesters containing sodium sulfonate. Also suitable are vinylpyrrolidone/ethyl methacrylate/methacrylic acid copolymers, as are sold by Stepan under the names Stepanhold-Extra and —R1, and the Carboset® grades from BF Goodrich.

Suitable cationic polymers are, for example, cationic polymers with the INCI name Polyquaternium, e.g. copolymers of vinylpyrrolidone/N-vinylimidazolium salts (Luviquat® FC, Luviquat® HM, Luviquat® MS, Luviset Clear®, Luviquat Supreme®, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by the reaction of polyvinylpyrrolidone with quaternary ammonium compounds), polymer JR (hydroxyethylcellulose with cationic groups) and plant-based cationic polymers, e.g. guar polymers, such as the Jaguar® grades from Rhodia.

Very particularly suitable polymers are neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethylenimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF SE), or polyvinylpyrrolidone and copolymers thereof, in particular with vinyl esters, such as vinyl acetate, e.g. Luviskol® VA 37, VA 55, VA 64, VA 73 (BASF SE); polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers available under the name Amphomer® (National Starch), and zwitterionic polymers, as are described, for example, in the German patent applications DE 39 29 973, DE 21 50 557, DE 28 17 369 and DE 37 08 451.

Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

In one specific embodiment, the compositions according to the invention comprise at least one polymer which differs from the polymers present in the copolymer compositions A) and which acts as thickener.

Suitable polymeric thickeners are, for example, optionally modified polymeric natural substances (carboxymethylcellulose and other cellulose ethers, hydroxyethyl- and -propylcellulose and the like) and also synthetic polymeric thickeners (polyacrylic and polymethacrylic compounds, vinyl polymers, polycarboxylic acids, polyethers, polyimines, polyamides). These include the in part already aforementioned polyacrylic and polymethacrylic compounds, for example the high molecular weight ones with a polyalkenyl polyether, in particular an allyl ether of sucrose, pentaerythritol or propylene, crosslinked homopolymers of acrylic acid (INCI name: carbomer). Such polyacrylic acids are available, inter alia, from BF Goodrich under the trade name Carbopol®, e.g. Carbopol 940 (molecular weight ca. 4 000 000), Carbopol 941 (molecular weight ca. 1 250 000) or Carbopol 934 (molecular weight ca. 3 000 000). Acrylic acid copolymers, which are available, for example, from Rohm & Haas under the trade names Aculyn® and Acusol®, also include, for example, the anionic, nonassociative polymers Aculyn 22, Aculyn® 28, Aculyn 33 (crosslinked), Acusol 810, Acusol 823 and Acusol 830 (CAS 25852-37-3). Also specifically suitable are associative thickeners, e.g. based on modified polyurethanes (HEUR) or hydrophobically modified acrylic acid or methacrylic acid copolymers (HASE thickeners, High Alkali Swellable Emulsion).

The use amount of the additional thickeners is preferably in a range from 0.001 to 10% by weight, preferably 0.1 to 5%, based on the total weight of the composition.

Examples of effect substances which can be formulated as aqueous active ingredient composition according to the invention are dyes: e.g. the dyes described in DE-A 102 45 209, and also the compounds referred to according to the Colour Index as disperse dyes and as solvent dyes, which are also referred to as dispersion dyes. A list of suitable dispersion dyes can be found, for example, in Ullmanns Enzyklopädie der technischen Chemie [Ullmann's encyclopedia of industrial chemistry], 4th edition, vol. 10, pp. 155-165 (see also vol. 7, p. 585ff-Anthraquinone dyes; vol. 8, p. 244ff-Azo dyes; vol. 9, p. 313ff-Quinophthalone dyes). Reference is hereby expressly made to this reference and the compounds cited therein. Dispersion dyes and solvent dyes suitable according to the invention comprise highly different dye classes with varying chromophores, for example anthraquinone dyes, monoazo and disazo dyes, quinophthalones, methine and azamethine dyes, naphthalimide dyes, naphthoquinone dyes and nitro dyes. Examples of dispersion dyes suitable according to the invention are the dispersion dyes of the following Colour Index list: C. I. Disperse Yellow 1-228,

C. I. Disperse Orange 1-148, C. I. Disperse Red 1-349, C. I. Disperse Violet 1-97, C. I. Disperse Blue 1-349, C. I. Disperse Green 1-9, C. I. Disperse Brown 1-21, C. I. Disperse Black 1-36. Examples of solvent dyes suitable according to the invention are the compounds of the following Colour Index list: C. I. Solvent Yellow 2-191, C. I. Solvent Orange 1-113, C. I. Solvent Red 1-248, C. I. Solvent Violet 2-61, C. I. Solvent Blue 2-143, C. I. Solvent Green 1-35, C. I. Solvent Brown 1-63, C. I. Solvent Black 3-50. Dyes suitable according to the invention are also derivatives of naphthalene, of anthracene, of perylene, of terylene, of quarterylene, and also diketopyrrolopyrrole dyes, perinone dyes, coumarin dyes, isoindoline and isoindolinone dyes, porphyrin dyes, phthalocyanine and naphthalocyanine dyes.

Besides the aforementioned constituents, the active ingredient and effect substance compositions according to the invention can also comprise conventional surface-active substances and other additives. The surface-active substances include surfactants, dispersion auxiliaries and wetting agents. The other additives include, in particular, thickeners, antifoams, preservatives, antifreezes, stabilizing agents, etc.

In principle, it is possible to use anionic, cationic, nonionic and amphoteric surfactants, with polymer surfactants and also surfactants with heteroatoms being included in the hydrophobic group.

The anionic surfactants include, for example, carboxylates, in particular alkali metal, alkaline earth metal and ammonium salts of fatty acids, e.g. potassium stearate, which are usually also referred to as soaps; acyl glutamates; sarcosinates, e.g. sodium lauroyl sarcosinate; taurates; methylcelluloses; alkyl phosphates, in particular mono- and diphosphoric acid alkyl esters; sulfates, in particular alkyl sulfates and alkyl ether sulfates; sulfonates, further alkyl- and alkylarylsulfonates, in particular alkali metal, alkaline earth metal and ammonium salts of arylsulfonic acids, and also alkyl-substituted arylsulfonic acids, alkylbenzenesulfonic acids, such as, for example, ligno- and phenolsulfonic acid, naphthalene- and dibutylnaphthalenesulfonic acids, or dodecylbenzenesulfonates, alkylnaphthalenesulfonates, alkyl methyl ester sulfonates, condensation products of sulfonated naphthalene and derivatives thereof with formaldehyde, condensation products of naphthalenesulfonic acids, phenolic and/or phenolsulfonic acids with formaldehyde or with formaldehyde and urea, mono- or dialkylsuccinic acid ester sulfonates; and protein hydrolyzates and lignosulfite waste liquors. The aforementioned sulfonic acids are advantageously used in the form of their neutral or optionally basic salts.

The cationic surfactants include, for example, quaternized ammonium compounds, in particular alkyltrimethylammonium and dialkyldimethylammonium halides and alkyl sulfates, and also pyridine and imidazoline derivatives, in particular alkylpyridinium halides.

The nonionic surfactants include, for example:
fatty alcohol polyoxyethylene esters, for example lauryl alcohol polyoxyethylene ether acetate,
alkyl polyoxyethylene and polyoxypropylene ethers, e.g. of isotridecyl alcohol and fatty alcohol polyoxyethylene ethers,
alkylaryl alcohol polyoxyethylene ethers, e.g. octylphenol polyoxyethylene ether,
alkoxylated animal and/or vegetable fats and/or oils, for example corn oil ethoxylates, castor oil ethoxylates, tallow fatty ethoxylates,
glycerol esters, such as, for example, glycerol monostearate,
fatty alcohol alkoxylates and oxo alcohol alkoxylates, in particular of the type RO—(R₁₈O)ᵣ(R₁₉O)ₛR₂₀ where R₁₈ and R₁₉ independently of one another ═C₂H₄, C₃H₆, C₄H and R₂₀═H or C₁-C₁₂-alkyl, R═C₃-C₃₀-alkyl or C₆-C₃₀-alkenyl, r and s independently of one another are 0 to 50, where both cannot be 0, such as isotridecyl alcohol and oleyl alcohol polyoxyethylene ether,
alkylphenol alkoxylates, such as, for example, ethoxylated isooctyl-, octyl- or nonylphenol, tributylphenol polyoxyethylene ether,
fatty amine alkoxylates, fatty acid amide and fatty acid diethanolamide alkoxylates, in particular their ethoxylates,
sugar surfactants, sorbitol esters, such as, for example, sorbitan fatty acid esters (sorbitan monooleate, sorbitan tristearate), polyoxyethylene sorbitan fatty acid esters, alkyl polyglycosides, N-alkylgluconamides,
alkyl methyl sulfoxides,
alkyl dimethylphosphine oxides, such as, for example, tetradecyl dimethylphosphine oxide.

The amphoteric surfactants include, for example, sulfobetaines, carboxybetaines and alkyldimethylamine oxides, e.g. tetradecyldimethylamine oxide.

Further surfactants which are to be specified here by way of example are perfluoro surfactants, silicone surfactants, phospholipids, such as, for example, lecithin or chemically modified lecithins, amino acid surfactants, e.g. N-lauroyl glutamate.

Unless specified, the alkyl chains in the surfactants listed above are linear or branched radicals having usually 8 to 20 carbon atoms.

The active ingredient or effect substance compositions according to the invention can comprise water-soluble salts as component B) and/or C), e.g. NaCl.

The active ingredient or effect substance compositions according to the invention can comprise organic solvents, oils and/or fats for some applications. Preference is given to those solvents, oils and/or fats which are environmentally compatible or biocompatible. These include, for example,
paraffin oils, aromatic hydrocarbons and aromatic hydrocarbon mixtures, e.g. xylenes, Solvesso 100, 150 or 200, and the like,
phenols and alkylphenols, e.g. phenol, hydroquinone, nonylphenol, etc.
ketones with more than 4 carbon atoms, such as cyclohexanone, isophorone, isopherone, acetophenone, acetonaphthone,
alcohols with more than 4 carbon atoms, such as acetylated lanolin alcohol, cetyl alcohol, 1-decanol, 1-heptanol, 1-hexanol, isooctadecanol, isopropyl alcohol, oleyl alcohol, benzyl alcohol,
carboxylic acid esters, e.g. dialkyl esters of adipic acid such as bis(2-ethylhexyl) adipate, dialkyl esters of phthalic acid such as bis(2-ethylhexyl) phthalate, alkyl esters of acetic acid (including branched alkyl groups) such as ethyl acetate and ethyl acetoacetate, stearates such as butyl stearate, glycerol monostearate, citrates such as acetyl tributyl citrate, also cetyl octanoate, methyl oleate, methyl p-hydroxybenzoate, methyl tetradecanoate, propyl p-hydroxybenzoate, methyl benzoate, lactic acid esters such as isopropyl lactate, butyl lactate and 2-ethylhexyl lactate,
vegetable oils such as palm oil, rapeseed oil, castor oil and derivatives thereof, such as, for example, oxidized ones, coconut oil, cod-liver oil, corn oil, soybean oil, linseed oil, olive oil, peanut oil, dyers safflower oil, sesame seed oil, grapefruit oil, basil oil, apricot oil, ginger oil, geranium oil, orange oil, rosemary oil, macadamia oil, onion oil, mandarin oil, pine oil, sunflower oil,
hydrogenated vegetable oils such as hydrogenated palm oil, hydrogenated rapeseed oil, hydrogenated soybean oil,
animal oils such as pork fat oil, fish oils,
dialkylamides of medium- to long-chain fatty acids, e.g. hallcomides, and also
vegetable oil esters, such as rapeseed oil methyl ester.

The copolymer compositions A) can be used together with conventional thickeners. These include the aforementioned polymers effective as thickeners. These further include polysaccharides and organic layer minerals such as Xanthan Gum® (Kelzan® from Kelco), Rhodopol® 23 (Rhone Poulenc) or Veegum® (R. T. Vanderbilt) or Attaclay® (Engelhardt). Suitable thickeners are also organic natural thickeners (agar agar, carrageen, tragacanth, gum arabic, alginates, pectins, polyoses, guar flour, carob seed flour, starch, dextrines, gelatine, caseine) and inorganic thickeners (polysilicic acids, clay minerals such as montmorillonites, zeolites, silicas). Further thickeners are the polysaccharides and heteropolysaccharides, in particular the polysaccharide gums, for example gum arabic, agar, alginates, carrageens and their salts, guar, guaran, tragacanth, gellan, ramsan, dextran or xanthan and their derivatives, e.g. propoxylated guar, and also their mixtures. Other polysaccharide thickeners are, for example, starches of highly diverse origin and starch derivatives, e.g. hydroxyethyl starch, starch phosphate esters or starch acetates, or carboxymethylcellulose or its sodium salt, methyl-, ethyl-, hydroxyethyl-, hydroxypropyl-, hydroxypropylmethyl- or hydroxyethylmethylcellulose or cellulose acetate. Thickeners which can be used are also sheet silicates. These include, for example, the magnesium or sodium-magnesium layered silicates from Solvay Alkali available under the trade name Laponite®, and also the magnesium silicates from Sud-Chemie.

The use amount of the additional thickeners is preferably in a range from 0.001 to 10% by weight, preferably 0.1 to 5%, based on the total weight of the composition.

Suitable antifoams suitable for dispersions according to the invention are, for example, silicone emulsions (such as, for example, Silikon® SRE, Wacker or Rhodorsil® from Rhodia), long-chain alcohols, fatty acids, organofluorine compounds and mixtures thereof.

Bactericides can be added to stabilize the compositions according to the invention against infestation of microorganisms. Suitable bactericides are, for example, Proxel® from ICI or Acticide® RS from Thor Chemie and Kathon® MK from Rohm & Haas.

Suitable antifreezes are organic polyols, e.g. ethylene glycol, propylene glycol or glycerol. These are usually used in amounts of not more than 10% by weight, based on the total weight of the active ingredient composition, so as not to exceed the desired content of volatile compounds. In one embodiment of the invention, the fraction of volatile organic compounds different therefrom is preferably not more than 1% by weight, in particular not more than 1000 ppm.

The active ingredient compositions according to the invention can optionally comprise 1 to 5% by weight of buffer, based on the total amount of the prepared formulation, for regulating the pH, the amount and type of buffer used being governed by the chemical properties of the active ingredient or active ingredients. Examples of buffers are alkali metal salts of weak inorganic or organic acids, such as, for example, phosphoric acid, boric acid, acetic acid, propionic acid, citric acid, fumaric acid, tartaric acid, oxalic acid or succinic acid.

In one particularly preferred embodiment, the copolymers according to the invention are used as component in a cosmetic composition. As described previously, they can serve here to modify the rheological properties of a cosmetic composition based on an aqueous medium.

The invention further provides a cosmetic composition comprising
A) at least one copolymer composition obtainable by a process according to the invention,
B) at least one cosmetically acceptable active ingredient and
C) optionally at least one further cosmetically acceptable auxiliary different from A) and B).

Preferably, component C) comprises at least one cosmetically or pharmaceutically acceptable carrier.

The carrier component C) is preferably selected from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols that are different from iii),
v) saturated acyclic and cyclic hydrocarbons,
vi) fatty acids,
vii) fatty alcohols,
viii) propellant gases,
   and mixtures thereof.

Suitable hydrophilic components C) are the aforementioned organic solvents, oils and fats.

Specifically suitable cosmetically compatible oil and fat components C) are described in Karl-Heinz Schrader, Grundlagen and Rezepturen der Kosmetika [Fundamentals and formulations of cosmetics], 2nd edition, Verlag Hüthig, Heidelberg, pp. 319-355, to which reference is made here.

The cosmetic compositions according to the invention may be skin cosmetic, hair cosmetic, dermatological, hygiene or pharmaceutical compositions. On account of their thickening properties, the copolymer compositions A) described above are suitable in particular as additives for hair and skin cosmetics. They are suitable specifically for the formulation of gels.

The compositions according to the invention are preferably in the form of a gel, foam, spray, salve, cream, emulsion, suspension, lotion, milk or paste. If desired, liposomes or microspheres can also be used.

The cosmetically active compositions according to the invention can additionally comprise cosmetically and/or dermatologically active ingredients and effect substances and also auxiliaries. Of suitability in principle are the aforementioned active ingredients and effect substances B) and also auxiliaries C).

The cosmetic compositions according to the invention preferably comprise at least one copolymer composition A) as defined above, at least one carrier C) as defined above and at least one constituent different therefrom which is preferably selected from cosmetically active ingredients, emulsifiers, surfactants, preservatives, perfume oils, additional thickeners, hair polymers, hair and skin conditioners, graft polymers, water-soluble or dispersible silicone-containing polymers, photoprotective agents, bleaches, gel formers, care agents, tinting agents, tanning agents, dyes, pigments, consistency regulators, humectants, refatting agents, collagen, protein hydrolyzates, lipids, antioxidants, antifoams, antistats, emollients and softeners.

In addition to the copolymer compositions A), conventional thickeners suitable for use in cosmetic compositions are those mentioned above.

Suitable cosmetically and/or dermatologically active ingredients are, for example, skin and hair pigmentation agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, active ingredients which have an antioxidative effect and/or free-radical scavenging effect, skin-moisturizing or -humectant substances, refatting active ingredients, deodorizing active ingredients, sebostatic active ingredients, plant extracts, antierythimatous or antiallergic active ingredients and mixtures thereof.

Artificially skin-tanning active ingredients which are suitable for tanning of the skin without natural or artificial irradiation with UV rays are, for example, dihydroxyacetone, alloxan and walnut shell extract. Suitable keratin-hardening substances are generally active ingredients as are also used in antiperspirants, such as, for example, potassium aluminum sulfate, aluminum hydroxychloride, aluminum lactate, etc. Antimicrobial active ingredients are used in order to destroy microorganisms and/or to inhibit their growth and thus serve both as preservatives and also as deodorizing substance which reduces the development or the intensity of body odor. These include, for example, customary preservatives known to the person skilled in the art, such as p-hydroxybenzoic acid esters, imidazolidinylurea, formaldehyde, sorbic acid, benzoic acid, salicylic acid, etc. Deodorizing substances of this type are, for example, zinc ricinoleate, triclosan, undecylenic acid alkylolamides, triethyl citrate, chlorhexidine etc. Suitable photofilter active ingredients are substances which absorb UV rays in the UV-B and/or UV-A region. Suitable UV filters are those specified above. Also suitable are p-aminobenzoic acid esters, cinnamic acid esters, benzophenones, camphor derivatives and pigments which stop UV rays, such as titanium dioxide, talc and zinc oxide. Suitable repellent active ingredients are compounds which are able to deter or drive away certain animals, in particular insects, from people. These include, for example, 2-ethyl-1,3-hexanediol, N,N-diethyl-m-toluamide etc. Suitable hyperemic substances, which stimulate blood flow through the skin, are, for example, essential oils, such as dwarf-pine, lavender, rosemary, juniper berry, horse chestnut extract, birch leaf extract, hay flower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camille extract, panthenol, etc.

The cosmetic compositions according to the invention can comprise, as cosmetic active ingredient (and also optionally as auxiliary), at least one cosmetically or pharmaceutically acceptable polymer which differs from the copolymers A) according to the invention. These include, very generally, anionic, cationic, amphoteric and neutral polymers. Reference is made here to the aforementioned polymers in their entirety.

According to a preferred embodiment, the compositions according to the invention are a skin cleaning composition.

Preferred skin cleaning compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oil baths and scrub preparations, shaving foams, shaving lotions and shaving creams.

According to a further preferred embodiment, the compositions according to the invention are cosmetic compositions for the care and protection of the skin, nail care compositions and preparations for decorative cosmetics.

Suitable skin cosmetic compositions are, for example, face toners, face masks, deodorants and other cosmetic lotions. Compositions for use in decorative cosmetics include, for example, concealing sticks, stage make-up, mascara and eye-shadows, lipsticks, kohl pencils, eyeliners, blushers, powders and eyebrow pencils.

Moreover, the copolymer compositions A) can be used in nose strips for pore cleansing, in antiacne compositions, repellents, shaving compositions, hair removal compositions, intimate care compositions, foot care compositions and also in baby care.

The skin care compositions according to the invention are in particular W/O or O/W skin creams, day and night creams, eye creams, face creams, antiwrinkle creams, moisturizing creams, bleaching creams, vitamin creams, skin lotions, care lotions and moisturizing lotions.

Skin cosmetic and dermatological compositions based on the above-described copolymer A) exhibit advantageous effects. The polymers can contribute, inter alia, to the moisture retention and conditioning of the skin and to improving the skin feel. By adding the polymers according to the invention, a considerable improvement in skin compatibility can be achieved in certain formulations.

Skin cosmetic and dermatological compositions comprise preferably at least one copolymer composition A) in a fraction of from about 0.001 to 30% by weight, preferably 0.01 to 20% by weight, very particularly preferably 0.1 to 12% by weight, based on the total weight of the composition.

Depending on the field of use, the compositions according to the invention can be applied in a form suitable for skin care, such as, for example, as cream, foam, gel, stick, mousse, milk, spray (pump spray or propellant-containing spray) or lotion.

Besides the copolymer compositions A) and suitable carriers, the skin cosmetic preparations can also comprise further active ingredients and auxiliaries customary in skin cosmetics, as described above. These include preferably emulsifiers, preservatives, perfume oils, cosmetic active ingredients such as phytantriol, vitamin A, E and C, retinol, bisabolol, panthenol, photoprotective agents, bleaches, tanning agents, collagen, protein hydrolyzates, stabilizers, pH regulators, dyes, salts, thickeners, gel formers, consistency regulators, silicones, humectants, refatting agents and further customary additives.

Preferred oil and fat components of the skin cosmetic and dermatological compositions are the aforementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with conventional polymers, as described above, if specific properties are to be set.

To set certain properties, such as, for example, improving the feel to the touch, the spreading behavior, the water resistance and/or the binding of active ingredients and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The preparation of the cosmetic or dermatological preparations takes place in accordance with customary processes known to the person skilled in the art.

The cosmetic and dermatological compositions are preferably in the form of emulsions, in particular as water-in-oil (W/O) or oil-in-water (O/W) emulsions. However, it is also possible to select other types of formulation, for example hydrodispersions, gels, oils, oleogels, multiple emulsions, for example in the form of W/O/W or O/W/O emulsions, anhydrous salves or salve bases, etc.

The preparation of emulsions takes place by known methods. Besides at least one copolymer composition A), the emulsions generally comprise customary constituents, such as fatty alcohols, fatty acid esters and in particular fatty acid triglycerides, fatty acids, lanolin and derivatives thereof, natural or synthetic oils or waxes and emulsifiers in the presence of water. The selection of the additives specific to the type of emulsion and the preparation of suitable emulsions is described, for example, in Schrader, Grundlagen and Rezepturen der Kosmetika [Fundamentals and formulations of cosmetics], Hüthig Buch Verlag, Heidelberg, 2nd edition, 1989, third part, to which reference is hereby expressly made.

A suitable emulsion, e.g. for a skin cream etc., generally comprises an aqueous phase emulsified by means of a suitable emulsifier system in an oil or fat phase. A copolymer composition A) can be used to provide the aqueous phase.

Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil; mineral oils whose distillation start-point under atmospheric pressure is ca. 250° C. and whose distillation end point is 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fatty phase may also comprise silicone oils that are soluble in other oils, such as dimethylpolysiloxane, methylphenylpolysiloxane and the silicone glycol copolymer, fatty acids and fatty alcohols.

Besides the copolymer compositions A), waxes can also be used, such as, for example, carnauba wax, candililla wax, beeswax, microcrystalline wax, ozokerite wax and Ca, Mg and Al oleates, myristates, linoleates and stearates.

Furthermore, an emulsion according to the invention may be present as O/W emulsion. An emulsion of this type usually comprises an oil phase, emulsifiers which stabilize the oil phase in the water phase, and an aqueous phase which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one copolymer composition A) and usually anionic surfactants as base surfactants and amphoteric and/or nonionic surfactants as cosurfactants. Further suitable active ingredients and/or auxiliaries are generally selected from lipids, perfume oils, dyes, organic acids, preservatives and antioxidants and also thickeners/gel formers, skin conditioners and humectants.

These formulations preferably comprise 2 to 50% by weight, preferably 5 to 40% by weight, particularly preferably 8 to 30% by weight, of surfactants, based on the total weight of the formulation.

All anionic, neutral, amphoteric or cationic surfactants customarily used in body cleaning compositions can be used in the washing, shower and bath preparations.

Suitable surfactants are those specified above.

Furthermore, the shower gel/shampoo formulations can comprise additional thickeners, such as, for example, sodium chloride, PEG-55, propylene glycol oleate, PEG-120 methylglucose dioleate and others, and also preservatives, further active ingredients and auxiliaries and water.

According to a further preferred embodiment, the compositions according to the invention are a hair treatment composition.

Hair treatment compositions according to the invention preferably comprise at least one copolymer composition A) in an amount in the range from about 0.1 to 30% by weight, preferably 0.5 to 20% by weight, based on the total weight of the composition.

The hair treatment compositions according to the invention are preferably present in the form of a setting foam, hair mousse, hair gel, hair shampoo, hairspray, hair foam, end fluid, neutralizer for permanent waves or “hot-oil treatments”. Depending on the field of use, the hair cosmetic preparations can be applied as (aerosol) spray, (aerosol) foam, gel, gel spray, cream, lotion or wax. Hairsprays here comprise both aerosol sprays and also pump sprays without propellant gas. Hair foams comprise both aerosol foams and also pump foams without propellant gas. Hairsprays and hair foams comprise preferably predominantly or exclusively water-soluble or water-dispersible components. If the compounds used in the hairsprays and hair foams according to the invention are water-dispersible, they can be applied in the form of aqueous microdispersions having particle diameters of usually 1 to 350 nm, preferably 1 to 250 nm. The solids contents of these preparations are usually in a range from about 0.5 to 20% by weight. These microdispersions generally require no emulsifiers or surfactants for their stabilization.

In a preferred embodiment, the hair cosmetic formulations according to the invention comprise
a) 0.05 to 5% by weight, preferably 0.1 to 3% by weight, of at least one copolymer composition A),
b) 20 to 99.95% by weight of water and/or alcohol,
c) 0 to 50% by weight of at least one propellant gas,
d) 0 to 5% by weight of at least one emulsifier,
e) 0 to 3% by weight of at least one thickener different from a), and
f) 0 to 20% by weight, preferably 0.1 to 10% by weight, of at least one water-soluble or water-dispersible polymer different from a) to e) and g),
g) 0 to 45% by weight, preferably 0.05 to 25% by weight, of further constituents,
   where the components a) to g) add up to 100% by weight.

Alcohol is to be understood as meaning all alcohols customary in cosmetics, e.g. ethanol, isopropanol, n-propanol.

Further constituents are to be understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances such as panthenol, collagen, vitamins, protein hydrolyzates, alpha- and beta-hydroxycarboxylic acids, stabilizers, pH regulators, dyes, viscosity regulators, gel formers, salts, humectants, refatting agents, complexing agents and further customary additives.

Furthermore, these include all styling and conditioning polymers known in cosmetics which can be used in combination with the polymers according to the invention if very specific properties are to be set.

Suitable conventional hair cosmetic polymers are, for example, the aforementioned cationic, anionic, neutral, nonionic and amphoteric polymers, to which reference is made here.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and amino-functional silicone compounds such as amodimethicones (CTFA).

The copolymer compositions according to the invention are suitable in particular as thickeners in hair styling preparations, in particular hair foams and hair gels.

Emulsifiers which can be used are all emulsifiers that are customarily used in hair foams. Suitable emulsifiers may be nonionic, cationic and anionic or amphoteric.

A preparation suitable according to the invention for styling gels can, for example, have the following composition:
a) 0.1 to 5% by weight of at least one copolymer composition A),
b) 0 to 5% by weight of at least one cosmetically acceptable water-soluble or water-dispersible hair setting polymer different from A),
c) 80 to 99.85% by weight of water and/or alcohol,
d) 0 to 1% by weight of a gel former different from A),
e) 0 to 20% by weight of further constituents.

Additional gel formers which can be used are all gel formers customary in cosmetics. Reference is made in this regard to the aforementioned conventional thickeners.

The copolymer compositions A) according to the invention are also suitable for shampoo formulations, which additionally comprise customary surfactants.

In the shampoo formulations, to achieve certain effects, customary conditioners can be used in combination with the copolymer compositions A). These include, for example, the aforementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). In addition, protein hydrolyzates can be used, as can conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds such as amodimethicones (CTFA). In addition, cationic guar derivatives such as guar hydroxypropyltrimonium chloride (INCI) can be used.

The copolymer compositions A) to be used according to the invention are likewise suitable for the use for modifying the rheological properties in pharmaceutical preparations of any type.

The invention therefore further provides a pharmaceutical composition comprising
A) at least one copolymer composition as defined above,
B) at least one pharmaceutically acceptable active ingredient and
C) optionally at least one further pharmaceutically acceptable auxiliary different from A) and B).

The formulation base of the pharmaceutical compositions according to the invention preferably comprises pharmaceutically acceptable auxiliaries. Of pharmaceutical acceptability are the auxiliaries that are known for use in the field of pharmacy, food technology and related fields, in particular the auxiliaries listed in relevant pharmacopoeia (e.g. DAB, Ph. Eur., BP, NF), as well as other auxiliaries whose properties do not preclude a physiological use.

Suitable auxiliaries may be: glidants, wetting agents, emulsifying and suspending agents, preservatives, antioxidants, antiirritatives, chelating agents, emulsion stabilizers, film formers, gel formers, odor masking agents, resins, hydrocolloids, solvents, solubility promoters, neutralizing agents, permeation accelerators, pigments, quaternary ammonium compounds, refatting and superfatting agents, salve, cream or oil bases, silicone derivatives, stabilizers, sterilizing agents, propellants, drying agents, opacifiers, additional thickeners, waxes, softeners, white oils. An embodiment in this regard is based on specialist knowledge, as represented, for example, in Fiedler, H. P. Lexikon der Hilfsstoffe für Pharmazie, Kosmetik and angrenzende Gebiete [Lexicon of the auxiliaries for pharmacy, cosmetics and related fields], 4th edition, Aulendorf: ECV-Editio-Kantor-Verlag, 1996.

To prepare pharmaceutical compositions according to the invention, the active ingredients can be mixed or diluted with a suitable auxiliary (excipient). Excipients may be solid, semisolid or liquid materials which can serve as a vehicle, carrier or medium for the active ingredient. The admixing of further auxiliaries takes place if desired in the manner known to the person skilled in the art. In particular, these are aqueous solutions or solubilizates for oral or parenteral application. Furthermore, the copolymers to be used according to the invention are also suitable for use in oral administration forms such as tablets, capsules, powders, solutions. Here, they can provide the sparingly soluble medicament with increased bioavailability. In the case of parenteral application, emulsions, for example fatty emulsions, can also be used besides solubilizates.

Pharmaceutical formulations of the aforementioned type can be obtained by processing the copolymer compositions A) to be used according to the invention with pharmaceutical active ingredients by conventional methods and using known and new active ingredients.

The use according to the invention can additionally comprise pharmaceutical auxiliaries and/or diluents. Cosolvents, stabilizers, preservatives are particularly listed as auxiliaries.

The pharmaceutical active ingredients used are water-soluble substances or else insoluble or poorly soluble substances. According to DAB 9 (German pharmacopoeia), the grading of the solubility of pharmaceutical active ingredients is as follows: poorly soluble (soluble in 30 to 100 parts of solvent); sparingly soluble (soluble in 100 to 1000 parts of solvent); virtually insoluble (soluble in more than 10 000 parts of solvent). The active ingredients can come from any area of indication.

The content of copolymer A) in the pharmaceutical compositions is, depending on the active ingredient, in the range from 0.01 to 50% by weight, preferably 0.1 to 40% by weight, particularly preferably 1 to 30% by weight, based on the total weight of the composition.

Of suitability for preparing the pharmaceutical compositions according to the invention are in principle all pharmaceutical active ingredients and prodrugs. These include benzodiazepines, antihypertensives, vitamins, cytostatics—in particular taxol, anesthetics, neuroleptics, antidepressants, antibiotics, antimycotics, fungicides, chemotherapeutics, urologics, platelet aggregation inhibitors, sulfonamides, spasmolytics, hormones, immunoglobulins, sera, thyroid therapeutics, psychopharmaceuticals, antiparkinson agents and other antihyperkinetics, ophthalmics, neuropathy products, calcium metabolism regulators, muscle relaxants, narcotics, lipid-lowering agents, liver therapeutics, coronary agents, cardiac agents, immunotherapeutics, regulatory peptides and their inhibitors, hypnotics, sedatives, gynecologicals, antigout agents, fibrinolytics, enzyme products and transport proteins, enzyme inhibitors, emetics, perfusion promoters, diuretics, diagnostics, corticoids, cholinergics, binary therapeutics, antiasthmatics, broncholytics, beta-receptor blockers, calcium antagonists, ACE inhibitors, arteriosclerosis remedies, antiphlogistics, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, and weight-reduction agents. Examples of suitable pharmaceutical active ingredients are the active ingredients specified in particular in paragraphs 0105 to 0131 of US 2003/0157170.

Besides the use in cosmetics and in pharmacy, the copolymer compositions A) to be used according to the invention are also suitable in the food sector for modifying rheological properties. The invention therefore also provides food preparations which comprise at least one of the copolymer compositions A) to be used according to the invention. Within the context of the present invention, food preparations are also to be understood as meaning food supplements, such as, for example, preparations comprising food dyes and dietetic foods. Moreover, the specified copolymer compositions A) are also suitable for modifying the rheological properties of feed additives for animal nutrition.

Moreover, the copolymer compositions A) are suitable for preparing aqueous preparations of food supplements, such as water-insoluble vitamins and provitamins such as vitamin A, vitamin A acetate, vitamin D, vitamin E, tocopherol derivatives such as tocopherol acetate and vitamin K.

The invention further provides the use of a copolymer composition A), as defined above, as auxiliary in pharmacy, preferably as or in (a) coating composition(s) for solid medicament forms, for modifying rheological properties, as surface-active compound, as or in (an) adhesive(s) and as or in (a) coating composition(s) for the textile, paper, printing and leather industry.

The invention is illustrated in more detail by reference to the following nonlimiting examples.

### EXAMPLES

### General Process A

(Examples 2, 3 and 4 in table I)

| | | | |
|---|---|---|---|
| Initial charge | Cyclohexane | 350 | g |
| | Ethyl acetate | 350 | g |
| | Beeswax 8104 (Kahl)^{#} | 5 | g |
| | Glycerol monostearate | 20 | g |
| | Wako ® V65* | 0.1 | g |
| | tert-Butyl peroctoate | 2.0 | g |
| | Feed 1 | 250 | g |
| Feed 1 | Cyclohexane | 180 | g |
| | Ethyl acetate | 180 | g |
| | Acrylic acid | 497.5 | g |
| | Pentaerythritol triallyl ether | 2.5 | g |
| Feed 2 | Cyclohexane | 140 | g |
| | Ethyl acetate | 140 | g |
| | Wako ® V65* | 0.15 | g |
| | tert-Butyl peroctoate | 2.0 | g |
| Feed 3 | Cyclohexane | 370 | g |
| | Ethyl acetate | 370 | g |

^{#}Beeswax 8104 obtainable from Kahl GmbH & Co. KG, Germany
*Wako ® V65 azobis(2,4-dimethylvaleronitrile), obtainable from Wako Chemicals

### Reaction Procedure:

The initial charge was heated to ca. 60° C. in a pressurized apparatus fitted with reflux condenser, internal thermometer and three separate feed devices while passing N2 through and stirring. The remainder of feed 1 was then metered in over 4 h, during which the first clouding was observed over the course of about 15 minutes. 30 minutes after the start of the addition of feed 1, feed 2 was metered in over 6 h. 60 minutes after the start of the addition of feed 1, feed 3 was metered in over 5 h. When the addition of feed 2 was complete, the reaction mixture was heated to 75° C. and stirred at 75° C. for 3 h. The reaction mixture was then heated to 90° C. and after-polymerized at 90° C. over a period of 5 h.

The product obtained was filtered off and dried in a drying cabinet at ca. 70° C. for 20 h. In example 4, the filtered-off product was additionally slurried once in a mixture of cyclohexane and ethyl acetate (1:1) and thereby washed, filtered off and then dried in the drying cabinet at ca. 70° C. for 20 h. In example 5, the filtered-off product was additionally slurried twice in a mixture of cyclohexane and ethyl acetate (1:1), filtered off and then finally dried in the drying cabinet at ca. 70° C. for 20 h.

The copolymer compositions of examples 1 to 4 and 6 to 17 in table I were prepared in an analogous manner:

The copolymer compositions of comparative examples V1, V2, V3 and V4 were also prepared according to process A. In contrast to the copolymer compositions prepared according to the invention, in the case of the comparison compositions V1, V2 and V3, the solvent was separated off by evaporation. Moreover, in comparative examples V1, V2, V3 and V4 a considerable amount of deposit was always formed, which led to poor heat transfer in the apparatus.

### General Process B

(Example 5 in table I)

| | | | |
|---|---|---|---|
| Initial charge | Cyclohexane | 350 | g |
| | Ethyl acetate | 350 | g |
| | Beeswax 8104 (Kahl)¹⁾ | 5 | g |
| | Glycerol monostearate | 20 | g |
| | Wako ® V65²⁾ | 0.1 | g |
| | tert-Butyl peroctoate | 2.0 | g |
| | Feed 1 | 250 | g |
| Feed 1 | Cyclohexane | 180 | g |
| | Ethyl acetate | 180 | g |
| | Acrylic acid | 497.5 | g |
| | Pentaerythritol triallyl ether | 2.5 | g |
| Feed 2 | Cyclohexane | 140 | g |
| | Ethyl acetate | 140 | g |
| | Wako ® V65* | 0.15 | g |
| | tert-Butyl peroctoate | 2.0 | g |
| Feed 3 | Cyclohexane | 250 | g |
| | Ethyl acetate | 250 | g |
| Feed 4 | Cyclohexane | 120 | g |
| | Ethyl acetate | 120 | g |
| | Tween ® 20³⁾ | 25 | g |

¹⁾Beeswax 8104 obtainable from Kahl GmbH & Co. KG, Germany
²⁾Wako ® V65 azobis(2,4-dimethylvaleronitrile), obtainable from Wako Chemicals
³⁾Tween ® 20 polyoxyethylene(20) sorbitan monolaurate

### Reaction Procedure:

The initial charge was heated to ca. 60° C. in a pressurized apparatus with reflux condenser, internal thermometer and three separate feed devices while passing N2 through and with stirring. The remainder of feed 1 was metered in over 4 h, during which the first clouding was observed over the course of about 15 minutes. 30 minutes after the start of feed 1, feed 2 was metered in over 6 h. 60 minutes after the start of feed 1, feed 3 was metered in over 4 h. Feed 4 is then metered in over the course of 90 minutes. When the addition of feed 4 was complete, the reaction mixture was heated to 75° C. and after-stirred at 75° C. for 3 h. The reaction mixture was then heated to 90° C. and after-polymerized at 90° C. for 4 h.

The product obtained was filtered off and dried in a drying cabinet at ca. 70° C. for 20 h.

### General Process C

(Example 19 in table I)

| | | | |
|---|---|---|---|
| Initial charge | Cyclohexane | 350 | g |
| | Ethyl acetate | 350 | g |
| | Beeswax 8104 (Kahl)¹⁾ | 5 | g |
| | Glycerol monostearate | 20 | g |
| | Wako ® V65²⁾ | 0.1 | g |
| | tert-Butyl peroctoate | 2.0 | g |
| | Feed 1 | 150 | g |
| Feed 1 | Cyclohexane | 180 | g |
| | Ethyl acetate | 180 | g |
| | Acrylic acid | 347.5 | g |
| | Vinylpyrrolidone | 90 | g |
| | Vinylimidazole | 10 | g |
| | Plex-6877-O³⁾ | 50 | g |
| | Pentaerythritol triallyl ether | 2.5 | g |
| | Lutensol ® XL-70⁴⁾ | 25 | g |
| Feed 2 | Cyclohexane | 140 | g |
| | Ethyl acetate | 140 | g |
| | Wako ® V65²⁾ | 0.15 | g |
| | tert-Butyl peroctoate | 2.0 | g |
| Feed 3 | Cyclohexane | 250 | g |
| | Ethyl acetate | 250 | g |

¹⁾Beeswax 8104 obtainable from Kahl GmbH & Co. KG, Germany
²⁾Wako ® V65 azobis(2,4-dimethylvaleronitrile), obtainable from Wako Chemicals
³⁾Plex-6877-O C₁₈₋₂₂ alkyl PEG methacrylate in MMA (25% strength)
⁴⁾Lutensol ® XL-70 C₁₀-Guerbet alcohol alkoxylate (7 EO)

### Reaction Procedure:

The initial charge was heated to ca. 60° C. in a pressurized apparatus fitted with reflux condenser, internal thermometer and three separate feed devices while passing N2 through and with stirring. The remainder of feed 1 was metered in over 4 h, during which the first clouding was observed over the course of about 30 minutes. 30 minutes after the start of feed 1, feed 2 was metered in over 6 h. 60 minutes after the start of feed 1, feed 3 was metered in over 5.5 h. After the end of feed 2, the reaction mixture was heated to 75° C. and after-stirred at 75° C. for 3 h. The reaction mixture was then heated to 90° C. and after-polymerized at 90° C. for 4 h.

The product obtained was filtered off and dried in a drying cabinet at ca. 70° C. for 20 h.

The copolymer compositions of examples 21 and 22 in table I were prepared in an analogous manner.

The copolymer compositions obtained according to processes A, B or C are listed in table I. The quantitative data in table I are (unless stated otherwise) in % by weight, based on the unsaturated monomers used for the polymerization. Table II lists data relating to the purification and product properties of the copolymer compositions.

TABLE I

| | | | | | | | h1 + h2 | | |
|---|---|---|---|---|---|---|---|---|---|
| | | | | Plex- | Lutencryl | | (GMS + | h3 | Process/SC [%] |
| Ex. | AA | VP | VI | 6877-O | AT 250 | PETAE | BW) [ppH] | [ppH] | in [CH:EE] |
| V1 | 99.5 | — | — | — | — | 0.5 | — | — | A |
| | | | | | | | | | 15% |
| | | | | | | | | | [50:50] |
| V2 | 99.5 | — | — | — | — | 0.5 | 5 + 0 | | A |
| | | | | | | | | | 18% |
| | | | | | | | | | [50:50] |
| V3 | 99.5 | — | — | — | — | 0.5 | 0 + 5 | | A |
| | | | | | | | | | 20% |
| | | | | | | | | | [50:50] |
| V4 | 99.5 | — | — | — | — | 0.5 | 0 + 5 | | A |
| | | | | | | | | | 20% |
| | | | | | | | | | [50:50] |
| 1 | 99.5 | — | — | — | — | 0.5 | 4.5 + 0.5 | | A |
| | | | | | | | | | 22% |
| | | | | | | | | | [50:50] |
| 2 | 99.5 | — | — | — | — | 0.5 | 4 + 1 | | A |
| | | | | | | | | | 24% |
| | | | | | | | | | [50:50] |
| 3 | 99.5 | — | — | — | — | 0.5 | 4 + 1 | | A |
| | | | | | | | | | 24% |
| | | | | | | | | | [50:50] |
| 4 | 99.5 | — | — | — | — | 0.5 | 4 + 1 | | A |
| | | | | | | | | | 24% |
| | | | | | | | | | [50:50] |
| 5 | 99.5 | — | — | — | — | 0.5 | 4 + 1 | 5* | B |
| | | | | | | | | Tween 20 | 24% |
| | | | | | | | | | [50:50] |
| 6 | 99.7 | — | — | — | — | 0.3 | 4 + 1 | | A |
| | | | | | | | | | 24% |
| | | | | | | | | | [50:50] |
| 7 | 99.3 | — | — | — | — | 0.7 | 4 + 1 | | A |
| | | | | | | | | | 24% |
| | | | | | | | | | [50:50] |
| 8 | 99.5 | — | — | — | — | 0.5 | 5 + 1 | | A |
| | | | | | | | | | 22% |
| | | | | | | | | | [47:53] |
| 9 | 99.5 | — | — | — | — | 0.5 | 4 + 2 | | A |
| | | | | | | | | | 22% |
| | | | | | | | | | [47:53] |
| 10 | 99.5 | — | — | — | — | 0.5 | 3 + 3 | | A |
| | | | | | | | | | 24% |
| | | | | | | | | | [47:53] |
| 11 | 99.5 | — | — | — | — | 0.5 | 2 + 4 | | A |
| | | | | | | | | | 24% |
| | | | | | | | | | [47:53] |
| 12 | 99.5 | — | — | — | — | 0.5 | 4 + 1 | | A |
| | | | | | | | | | 20% |
| | | | | | | | | | [60:40] |
| 13 | 99.5 | — | — | — | — | 0.5 | 4 + 1 | | A |
| | | | | | | | | | 24% |
| | | | | | | | | | [40:60] |
| 14 | 98.5 | — | 1 | — | — | 0.5 | 4 + 1 | | A |
| | | | | | | | | | 24% |
| | | | | | | | | | [47:53] |
| 15 | 94.6 | — | — | — | 5 | 0.4 | 4 + 1 | | A |
| | | | | | | | | | 20% |
| | | | | | | | | | [50:50] |
| 16 | 87.6 | — | 2 | 10 | | 0.4 | 4 + 1 | | A |
| | | | | | | | | | 20% |
| | | | | | | | | | [50:50] |
| 17 | 64.5 | 30 | — | | 5 | 0.5 | 1 + 0.5 | | A |
| | | | | | | | | | 20% |
| | | | | | | | | | [30:70] |
| 18 | 69.5 | 18 | 2 | — | — | 0.5 | 4 + 1 | 8* | B |
| | | | | | | | | XL-70 | 24% |
| | | | | | | | | | [30:70] |
| 19 | 69.5 | 18 | 2 | 10 | — | 0.5 | 4 + 1 | 5 | C |
| | | | | | | | | XL-70 | 24% |
| | | | | | | | | | [30:70] |
| 20 | 79.5 | 18 | 2 | — | — | 0.5 | 4 + 1 | 8* | B |
| | | | | | | | | XP-70 | 24% |
| | | | | | | | | | [47:53] |
| 21 | 79.5 | 18 | 2 | — | — | 0.5 | 4 + 1 | 5 | C |
| | | | | | | | | XP-70 | 24% |
| | | | | | | | | | [47:53] |
| 22 | 69.5 | 20 | | 10 | | 0.5 | 4 + 1 | 5 | C |
| | | | | | | | | XL-70 | 24 |
| | | | | | | | | | [20:80] |

All monomer data in % by weight
AA Acrylic acid
VP Vinylpyrrolidone
VI Vinylimidazole
Plex-6877-O C₁₈₋₂₂ alkyl PEG methacrylate in MMA (25% strength)
Lutencryl AT 250 C₁₆₋₁₈ alkyl PEG methacrylate in MAA (50% strength)
MMA Methyl methacrylate
MAA Methacrylic acid
PETAE Pentaerythritol triallyl ether
GMS Glycerol monostearate
BW Beeswax 8104 from KahI
XL-70 C₁₀-Guerbet alcohol alkoxylate (7 EO)
XP-70 C₁₀-Guerbet alcohol ethoxylate (7 EO)
SC Solids content
CH Cyclohexane
EE Ethyl acetate
ppH Parts per 100 parts of monomer
*Added after the polymerization
—

TABLE II
Process details/purification and product properties of the copolymer composition

| | | Viscosity [mPas] | |
|---|---|---|---|
| Ex. No. | Notes relating to the process | Appearance | Clarity |
| V1 | Very high viscosity of the reaction | 1) 8500 | Cloudy gel |
| | mixture; | 2) 32700 | |
| | considerable deposit formation in | | |
| | the apparatus leads to poor heat | | |
| | transfer; | | |
| | without purification; | | |
| V2 | High viscosity of the reaction | 1) 10500 | Cloudy gel (clarity 78%) |
| | mixture; | 2) 33400 | |
| | considerable deposit formation in | | |
| | the apparatus; | | |
| | without purification; | | |
| V3 | Considerable deposit formation in | 1) 10200 | Cloudy gel (clarity 81%) |
| | the apparatus; | 2) 36100 | |
| | without purification by means of | | |
| | filtration; | | |
| V4 | Considerable deposit formation in | 1) 11100 | Slightly cloudy gel (clarity 84%) |
| | the apparatus; | 2) 37500 | |
| | purification by means of filtration | | |
| 1 | Slight deposit formation on wall + | 1) 12500 | Virtually clear gel (clarity 89%) |
| | base of apparatus; | 2) 36850 | |
| | purification by means of filtration | | |
| 2 | Very slight deposit formation only | 1) 15500 | Virtually clear gel (clarity 90%) |
| | on the base; | 2) 39000 | |
| | purification by means of filtration | | |
| 3 | Very slight deposit formation only | 1) 13700 | Virtually clear gel (clarity 93%) |
| | on the base; | 2) 41200 | |
| | purification by means of 1 × | | |
| | slurrying CH/EE (1:1) and filtration | | |
| 4 | Very slight deposit formation only | 1) 14100 | Virtually clear gel (clarity 95%) |
| | on the base; | 2) 40800 | |
| | purification by means of 2 × | | |
| | slurrying CH/EE (1:1) and filtration | | |
| 5 | Very slight deposit formation only | Polymer particles with low | Virtually clear gel (clarity 90%) |
| | on the base; | pourability | |
| | purification by means of filtration | 1) 12700 | |
| | | 2) 41300 | |
| 6 | Very slight deposit formation only | 1) 7400 | Virtually clear gel (clarity 94%) |
| | on the base; | 2) 28300 | |
| | purification by means of 1 × | | |
| | filtration | | |
| 7 | Very slight deposit formation only | 1) 14500 | Virtually clear gel (clarity 89%) |
| | on the base; | 2) 32300 | |
| | purification by means of 1 × | | |
| | filtration | | |
| 8 | Very slight deposit formation only | 1) 12500 | Virtually clear gel (clarity 88%) |
| | on the base; | 2) 37900 | |
| | purification by means of 1 × | | |
| | filtration | | |
| 9 | Very slight deposit formation only | 1) 12700 | Virtually clear gel (clarity 89%) |
| | on the base; | 2) 38800 | |
| | purification by means of 1 × | | |
| | filtration | | |
| 10 | | | |
| 11 | | | |
| 12 | Very slight deposit formation only | 1) 11900 | Virtually clear gel (clarity 89%) |
| | on the base; | 2) 41000 | |
| | purification by means of 1 × | | |
| | filtration | | |
| 13 | Very slight deposit formation only | 1) 14200 | Virtually clear gel (clarity 91%) |
| | on the base; | 2) 43000 | |
| | purification by means of 1 × | | |
| | filtration | | |
| 14 | Very slight deposit formation only | 1) 12400 | Virtually clear gel (clarity 88%) |
| | on the base; | 2) 39100 | |
| | purification by means of 1 × | | |
| | filtration | | |
| 15 | Very slight deposit formation only | 1) 11400 | Virtually clear gel (clarity 87%) |
| | on the base; | 2) 34400 | |
| | purification by means of 1 × | | |
| | filtration | | |
| 16 | Very slight deposit formation only | 1) 12100 | Virtually clear gel (clarity 88%) |
| | on the base; | 2) 35200 | |
| | purification by means of 1 × | | |
| | filtration | | |
| 17 | Very slight deposit formation only | 1) 10400 | Virtually clear gel (clarity 90%) |
| | on the base; | 2) 34600 | |
| | purification by means of 1 × | | |
| | filtration | | |
| 18 | Very slight deposit formation only | Virtually dust-free polymer | Virtually clear gel (clarity 90%) |
| | on the base; | 1) 10400 | |
| | purification by means of 1 × | 2) 35300 | |
| | filtration | | |
| 19 | Lutensol XL-70 was firstly | 1) 11700 | Virtually clear gel (clarity 88%) |
| | dissolved in the monomers and | 2) 38600 | |
| | only then were these diluted with | | |
| | solvent; | | |
| | very slight deposit formation only | | |
| | on the base; | | |
| | purification by means of 1 × | | |
| | filtration | | |
| 20 | Very slight deposit formation only | 1) 12450 | Virtually clear gel (clarity 89%) |
| | on the base; | 2) 36300 | |
| | purification by means of 1 × | | |
| | slurrying CH/EE (1:1) and filtration | | |
| | purification by 1 × filtration | | |
| 21 | Lutensol XL-70 was firstly | 1) 10600 | Virtually clear gel (clarity 94%) |
| | dissolved in the monomers and | 2) 37200 | |
| | only then were these diluted with | | |
| | solvent; | | |
| | very slight deposit formation only | | |
| | on the base; | | |
| | purification by means of 1 × | | |
| | slurrying CH/EE (1:1) and filtration | | |
| | purification by 1 × filtration | | |

1) = 0.2% strength by weight aqueous solution
2) = 0.5% strength by weight aqueous solution
(Values determined by means of Brookfield viscometer at 23° C. and 100 s⁻¹)

1-31. (canceled)

32. A process for the preparation of a copolymer composition A) by free-radical copolymerization of a monomer composition comprising
a) acrylic acid,
b) optionally at least one compound, different from a), having a free-radically polymerizable, α,β-ethylenically unsaturated double bond and at least one anionogenic and/or anionic group per molecule,
c) at least one free-radically polymerizable crosslinking compound which comprises at least two α,β-ethylenically unsaturated double bonds per molecule,
by the method of precipitation polymerization in the presence of an auxiliary composition H) comprising
H1) glycerol monostearate, and
H2) at least one compound with an HLB value in the range from 4 to 10, selected from water-insoluble natural waxes, nonionic emulsifiers and mixtures thereof.

33. The process according to claim 32, where additionally at least one further auxiliary H3) is used which is selected from emulsifiers with an HLB value in the range greater than 10.

34. The process according to claim 32, where component b) comprises or consists of methacrylic acid.

35. The process according to claim 32, where the monomer composition used for the preparation of the copolymer composition A) additionally comprises at least one compound d) having a free-radically polymerizable, α,β-ethylenically unsaturated double bond and at least one cationogenic and/or cationic group per molecule.

36. The process according to claim 32, where the monomer composition used for the preparation of the copolymer composition A) additionally comprises at least one further monomer e) selected from α,β-ethylenically unsaturated amide-group-containing compounds of the general formula (II) 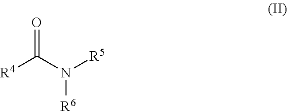
where
one of the radicals R⁴ to R⁶ is a group of the formula CH₂═CR⁷— where R⁷═H or C₁-C₄-alkyl and the other radicals R⁴ to R⁶, independently of one another, are H, alkyl, cycloalkyl, heterocycloalkyl, aryl or hetaryl,
where R⁴ and R⁵, together with the amide group to which they are bonded, may also be a lactam having 5 to 8 ring atoms,
where R⁵ and R⁶, together with the nitrogen atom to which they are bonded, may also be a five- to seven-membered heterocycle,
with the proviso that the sum of the carbon atoms of the radicals R⁴, R⁵ and R⁶ is at most 8.

37. The process according to claim 32, where the monomer composition used for the preparation of the copolymer composition A) additionally comprises, in copolymerized form, at least one further monomer f), different from the monomers e), which has a group of the formulae (Ma) or (Mb) 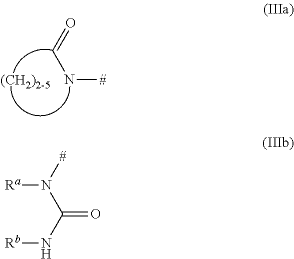
in which
# is the bonding site to a group with a free-radically polymerizable, α,β-ethylenically unsaturated double bond, where in the compounds (IIIa) # is not the bonding site to a group of the formula CH₂═CR⁷— where R⁷═H or C₁-C₄-alkyl,
R^{a} is H or C₁-C₄-alkyl,
R^{b} is H or C₁-C₄-alkyl, or
R^{a} and R^{b} together are (CH₂)₁₋₄.

38. The process according to claim 32, where the monomer composition used for the preparation of the copolymer composition A) additionally comprises, in copolymerized form, at least one further monomer g) which is selected from esters of α,β-ethylenically unsaturated mono- and dicarboxylic acids with C₁-C₇-alkanols, esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols which have a primary or secondary amino group, and mixtures thereof.

39. The process according to claim 32, where the monomer composition used for the preparation of the copolymer composition A) additionally comprises at least one compound h) which is selected from compounds of the general formulae IV a), IV b), IV c), IV d) and IV e) 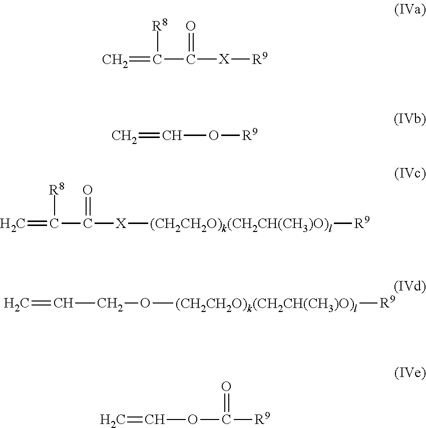
in which
the order of the alkylene oxide units is arbitrary,
k and l, independently of one another, are an integer from 0 to 1000, where the sum of k and l is at least 5,
R⁸ in the compounds (IVa) is hydrogen or C₁-C₄-alkyl, preferably methyl, and R⁸ in the compounds (IVc) is hydrogen or C₁-C₈-alkyl,
R⁹ in the compounds (IVa), (IVb) and (IVe) is C₈-C₃₀-alkyl or C₈-C₃₀-alkenyl, and R⁹ in the compounds (IVc) and (IVd) is hydrogen, C₁-C₃₀-alkyl, C₂-C₃₀-alkenyl or C₅-C₈-cycloalkyl,
X is O or a group of the formula NR¹⁰, in which R¹⁰ is H, alkyl, alkenyl, cycloalkyl, heterocycloalkyl, aryl or hetaryl.

40. The process according to claim 32, where the monomer composition used for the preparation of the copolymer composition A) additionally comprises at least one compound h) which is selected from urethane (meth)acrylates with alkylene oxide groups, esters of vinyl alcohol and allyl alcohol with C₁-C₇-monocarboxylic acids, C₁-C₇-alkyl vinyl ethers, vinylaromatics, vinyl halides, vinylidene halides, C₂-C₈-monoolefins, nonaromatic hydrocarbons with at least two conjugated double bonds and mixtures thereof.

41. The process according to claim 32, where, for the preparation of the copolymer composition A), based on the total weight of the monomers used for the polymerization,
98 to 99.9% by weight of acrylic acid a), and
0.1 to 2% by weight of at least one crosslinking compound c),
are used.

42. The process according to claim 32, where, for the preparation of the copolymer composition A), based on the total weight of the monomers used for the polymerization,
93 to 99.7% by weight of acrylic acid a),
0.2 to 5% by weight of at least one compound d) with a free-radically polymerizable, α,β-ethylenically unsaturated double bond and at least one cationogenic and/or cationic group per molecule, preferably vinylimidazole, and
0.1 to 2% by weight of at least one crosslinking compound c),
are used.

43. The process according to claim 32, where, for the preparation of the copolymer composition A), based on the total weight of the monomers used for the polymerization,
40 to 99.4% by weight of acrylic acid a),
0 to 40% by weight of at least one compound b), different from acrylic acid, having a free-radically polymerizable, α,β-ethylenically unsaturated double bond and at least one anionogenic and/or anionic group per molecule,
0.5 to 20% by weight of at least one compound g) and/or h), and
0.1 to 2% by weight of at least one crosslinking compound c),
are used, with the proviso that the total amount of the monomers a) and b) is 78 to 99.4% by weight.

44. The process according to claim 43, where, for the preparation of the copolymer composition A), based on the total weight of the monomers used for the polymerization,
40 to 99.4% by weight of acrylic acid a),
0 to 40% by weight of at least one compound b), different from acrylic acid, having a free-radically polymerizable α,β-ethylenically unsaturated double bond and at least one anionogenic and/or anionic group per molecule,
0 to 20% by weight of at least one monomer g) which is selected from C₁-C₇-alkyl(meth)acrylates, in particular methyl(meth)acrylate, ethyl(meth)acrylate, n-butyl(meth)acrylate and mixtures thereof,
0 to 20% by weight of at least one compound h) which is preferably selected from C₈-C₂₂-alkyl(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof, and
0.1 to 2% by weight of at least one crosslinking compound c),
are used, with the proviso that the total amount of the monomers a) and b) is 78 to 99.4% by weight and the sum of monomers g) and h) is 0.5 to 20% by weight.

45. The process according to claim 32, where, for the preparation of the copolymer composition A), based on the total weight of the monomers used for the polymerization,
48 to 99.4% by weight of acrylic acid a),
0 to 40% by weight of at least one compound b), different from acrylic acid, having a free-radically polymerizable α,β-ethylenically unsaturated double bond and at least one anionogenic and/or anionic group per molecule,
0.5 to 10% by weight, based on the total weight of a) and e), of at least one compound e) which is preferably selected from esters of α,β-ethylenically unsaturated mono- and dicarboxylic acids with C₈-C₃₀-alkanols, and
0.1 to 2% by weight of at least one crosslinking compound c),
are used, with the proviso that the total amount of the monomers a) and b) is 88 to 99.4% by weight.

46. The process according to claim 32, where, for the preparation of the copolymer composition A), based on the total weight of the monomers used for the polymerization,
40 to 99.4% by weight of acrylic acid a),
0 to 40% by weight of at least one compound b), different from acrylic acid, having a free-radically polymerizable α,β-ethylenically unsaturated double bond and at least one anionogenic and/or anionic group per molecule,
0 to 5% by weight of at least one compound d) with a free-radically polymerizable, α,β-ethylenically unsaturated double bond and at least one cationogenic and/or cationic group per molecule, preferably vinylimidazole,
0.1 to 30% by weight of at least one amide-group-containing compound e), preferably vinylpyrrolidone and/or vinylcaprolactam, and
0.1 to 2% by weight of at least one crosslinking compound c),
are used, with the proviso that the total amount of the monomers a) and b) is 65 to 99.8% by weight.

47. The process according to claim 46, where, for the preparation of the copolymer composition A), based on the total weight of the monomers used for the polymerization,
65 to 98.7% by weight of acrylic acid a),
0.2 to 5% by weight of vinylimidazole,
1 to 30% by weight of vinylpyrrolidone and/or vinylcaprolactam,
0.1 to 2% by weight of at least one crosslinking compound c),
are used.

48. The process according to claim 32, where the component H2) comprises or consists of a beeswax.

49. The process according to claim 32, where the polymerization takes place in an anhydrous, aprotic solvent or solvent mixture.

50. The process according to claim 49, where the polymerization takes place in a mixture of cyclohexane and ethyl acetate.

51. The process according to claim 32, where the auxiliaries H1) and/or H2) are initially introduced at least partly before the start of the polymerization.

52. The process according to claim 33, where the component H3) comprises or consists of a poly(oxyethylene) sorbitan monolaurate.

53. The process according to claim 33, where the auxiliary H3) is added after at least 90% of the monomers have reacted.

54. The process according to claim 32, where the copolymer composition A) is isolated after the precipitation polymerization and subjected to a washing with a liquid washing medium.

55. A copolymer composition A) obtainable by a process as defined in claim 32.

56. The use of a copolymer composition A) according to claim 55 or of a copolymer obtainable therefrom in an aqueous composition for modifying the rheological properties of this composition.

57. An active ingredient or effect substance composition comprising
A) at least one copolymer composition obtainable by a process as defined in claim 32,
B) at least one active ingredient or effect substance and
C) optionally at least one further auxiliary different from A) and B).

58. A cosmetic composition comprising
A) at least one copolymer composition obtainable by a process as defined in claim 32,
B) at least one cosmetically acceptable active ingredient and
C) optionally at least one further cosmetically acceptable auxiliary different from A) and B).

59. The composition according to claim 58 in the form of a gel.

60. A pharmaceutical composition comprising
A) at least one copolymer composition obtainable by a process as defined in claim 32,
B) at least one pharmaceutically acceptable active ingredient and
C) optionally at least one further pharmaceutically acceptable auxiliary different from A) and B).

61. The use of a copolymer composition A) obtainable by a process as defined in claim 32 in the food sector for modifying rheological properties.

62. The use of a copolymer composition A) obtainable by a process as defined in claim 32 as auxiliary in pharmacy, preferably as or in (a) coating composition(s) for solid medicament forms, for modifying rheological properties, as surface-active compound, as or in (an) adhesive(s) and as or in (a) coating composition(s) for the textile, paper, printing and leather industry.
